(19) United States
(12) Patent Application Publication
(10) Pub. No.: US 2023/0363362 A1
(43) Pub. Date: Nov. 16, 2023

(54) GENETICALLY MODIFIED NON-HUMAN ANIMAL WITH HUMAN OR CHIMERIC GLP1R

(71) Applicant: Biocytogen Pharmaceuticals (Beijing) Co., Ltd., Beijing (CN)

(72) Inventors: Yuelei Shen, Beijing (CN); Jiawei Yao, Beijing (CN); Yanan Guo, Beijing (CN); Yang Bai, Beijing (CN); Chengzhang Shang, Beijing (CN)

(21) Appl. No.: 18/025,990

(22) PCT Filed: Sep. 17, 2021
(86) PCT No.: PCT/CN2021/119112

§ 371 Date: Mar. 13, 2023

(30) Foreign Application Priority Data
Sep. 17, 2020 (CN) 202010979260.3
Jun. 25, 2021 (CN) 202110714328.X

(51) Int. Cl. A01K 67/027; C07K 14/72; C12N 15/85

(52) U.S. Cl. CPC A01K 67/0278; C07K 14/723; C12N 15/8509; A01K 2207/15; A01K 2217/072; A01K 2217/15; A01K 2227/105; A01K 2267/0362

(57) ABSTRACT

This disclosure relates to genetically modified animal expressing human or chimeric (e.g., humanized) GLP1R, and methods of use thereof.

### CLAIM OF PRIORITY

This application claims the benefit of Chinese Patent Application App. No. 202010979260.3, filed on Sep. 17, 2020 and Chinese Patent Application App. No. 202110714328.X, filed on Jun. 25, 2021. The entire contents of the foregoing are incorporated herein by reference.

### TECHNICAL FIELD

This disclosure relates to a genetically modified animal expressing human or chimeric (e.g., humanized) GLP1R, and methods of use thereof.

### BACKGROUND

GLP1R (Glucagon-like peptide-1 receptor) is a member of the B-type GPCR (G protein-coupled receptor) family, which is widely distributed in the islets, stomach, the small intestine, heart, kidney, brain and other tissues. Upon the binding of GLP-1 (Glucagon-like peptide-1, glucagon-like peptide-1), GLP1R activates downstream signaling pathways such as PKA, PI3K, MAPK, and participates in the release of insulin, R Cell proliferation, decreased glucagon, delayed gastric emptying, enhanced memory and other important physiological processes. It is a treatment target for type II diabetes. There are already a number of peptide drugs targeting this receptor on the market.

In view of the importance of GLP1R pathway, there is an urgent need to develop an animal model for testing therapies targeting GLP1R and its signaling pathway.

### SUMMARY

This disclosure is related to an animal model with human GLP1R or chimeric GLP1R. The animal model can express human GLP1R or chimeric GLP1R (e.g., humanized GLP1R) protein in its body. It can be used in the studies on the function of GLP1R gene, and can be used in the screening and evaluation of anti-human GLP1R antibodies. In addition, the animal models prepared by the methods described herein can be used in drug screening, pharmacodynamics studies, testing treatments for GLP1R related diseases. The disclosure also provides a powerful tool for studying the function of GLP1R protein and a platform for screening drugs.

In one aspect, the disclosure provides a genetically-modified, non-human animal whose genome comprises at least one chromosome comprising a sequence encoding a human or chimeric glucagon-like peptide-1 receptor (GLP1R). In some embodiments, the sequence encoding the human or chimeric GLP1R is operably linked to an endogenous regulatory element at the endogenous GLP1R gene locus in the at least one chromosome.

In some embodiments, the sequence encoding the human or chimeric GLP1R comprises a sequence encoding an amino acid sequence that is at least 70%, 75%, 80%, 85%, 90%, 95%, 99%, or 100% identical to SEQ ID NO: 2.

In some embodiments, the sequence encoding the human or chimeric GLP1R is operably linked to an endogenous 5′-UTR (e.g., immediately after 5′-UTR).

In some embodiments, the animal is a mammal, e.g., a monkey, a rodent or a mouse. In some embodiments, the animal is a mouse or a rat. In some embodiments, the animal does not express endogenous GLP1R or expresses a decreased level of endogenous GLP1R as compared to that of an animal without genetic modification.

In some embodiments, the animal has one or more cells expressing human or chimeric GLP1R. In some embodiments, the animal has one or more cells expressing human or chimeric GLP1R, and human glucagon-like peptide-1 (GLP-1) can bind to the expressed human or chimeric GLP1R. In some embodiments, the animal has one or more cells expressing human or chimeric GLP1R, and endogenous GLP1 can bind to the expressed human or chimeric GLP1R.

In one aspect, the disclosure provides a genetically-modified, non-human animal, wherein the genome of the animal comprises an insertion of a sequence encoding a region of human GLP1R at an endogenous GLP1R gene locus.

In some embodiments, the inserted sequence is operably linked to an endogenous regulatory element at the endogenous GLP1R locus, and one or more cells of the animal express human GLP1R or chimeric GLP1R.

In some embodiments, the animal does not express endogenous GLP1R or expresses a decreased level of endogenous GLP1R as compared to that of an animal without genetic modification.

In some embodiments, the inserted sequence is located immediately after 5′-UTR at the endogenous GLP1R locus.

In some embodiments, the animal has one or more cells expressing a chimeric GLP1R having one or more humanized extracellular regions, transmembrane regions, and cytoplasmic regions. In some embodiments, one or more of the humanized extracellular regions comprise a sequence that is at least 50%, 60%, 70%, 80%, 90%, 95%, or 99% identical to the corresponding extracellular region of human GLP1R.

In some embodiments, one or more of the humanized extracellular regions of the chimeric GLP1R has a sequence that has at least 10, 20, 30, 40, 50, 60, 70, 80, 90, or 95 contiguous amino acids that are identical to a contiguous sequence present in the corresponding extracellular region of human GLP1R.

In some embodiments, the animal further comprises a deletion of one or more nucleotide from the endogenous GLP1R gene.

In some embodiments, the animal further comprises an endogenous GLP1R 3′-UTR and/or a polyA sequence.

In some embodiments, the animal is heterozygous or homozygous with respect to the insertion at the endogenous GLP1R gene locus.

In one aspect, the disclosure provides a method for making a genetically-modified, non-human animal, comprising: inserting in at least one cell of the animal, at an endogenous GLP1R gene locus, a sequence encoding a region of human GLP1R gene.

In some embodiments, the sequence encoding the region of human GLP1R gene comprises exon 1, exon 2, exon 3, exon 4, exon 5, exon 6, exon 7, exon 8, exon 9, exon 10, exon 11, exon 12, and exon 13, or a part thereof, of a human GLP1R gene.

In some embodiments, the sequence encoding a region of human GLP1R gene encodes a sequence that is at least 90% identical to SEQ ID NO: 2.

In some embodiments, the animal is a mouse, and the endogenous GLP1R locus is within exon 1 of the mouse GLP1R gene.

In some embodiments, the animal further comprises deleting one or more nucleotides of the endogenous GLP1R gene.

In one aspect, the disclosure provides a non-human animal comprising at least one cell comprising a nucleotide sequence encoding a humanized GLP1R polypeptide. In some embodiments, the humanized GLP1R polypeptide comprises at least 50 contiguous amino acid residues that are identical to the corresponding contiguous amino acid sequence of a human GLP1R. In some embodiments, the animal expresses the humanized GLP1R.

In some embodiments, the humanized GLP1R polypeptide has at least 10 contiguous amino acid residues that are identical to the corresponding contiguous amino acid sequence of a human GLP1R extracellular region.

In some embodiments, the humanized GLP1R polypeptide comprises a sequence that is at least 90%, 95%, or 99% identical to SEQ ID NO: 2.

In some embodiments, the nucleotide sequence is operably linked to an endogenous GLP1R regulatory element of the animal (e.g., 5′-UTR).

In some embodiments, the humanized GLP1R polypeptide comprises one or more humanized extracellular regions, one or more humanized GLP1R transmembrane regions and/or one or more humanized GLP1R cytoplasmic regions.

In some embodiments, the nucleotide sequence is integrated to an endogenous GLP1R gene locus of the animal.

In one aspect, the disclosure provides a method of making a genetically-modified mouse cell that expresses a human GLP1R or a chimeric GLP1R, the method comprising: inserting at an endogenous mouse GLP1R gene locus, a nucleotide sequence encoding a human GLP1R or a chimeric GLP1R, thereby generating a genetically-modified mouse cell that includes a nucleotide sequence that encodes the human GLP1R or the chimeric GLP1R. In some embodiments, the mouse cell expresses the human GLP1R or the chimeric GLP1R.

In some embodiments, the entire coding sequence of human GLP1R gene is inserted at an endogenous mouse GLP1R gene locus.

In some embodiments, the chimeric GLP1R comprises or consists of one or more of the extracellular regions of human GLP1R; and one or more of the transmembrane regions; and/or one or more of the cytoplasmic regions of mouse GLP1R.

In some embodiments, the animal further comprises a sequence encoding an additional human or chimeric protein.

In some embodiments, the additional human or chimeric protein is glucagon-like peptide-1 (GLP1), cytotoxic T-lymphocyte-associated protein 4 (CTLA-4), Lymphocyte Activating 3 (LAG-3), B And T Lymphocyte Associated (BTLA), Programmed cell death protein 1 (PD-1), Programmed death-ligand 1 (PD-L1), CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (OX40).

In one aspect, the disclosure provides a method of determining effectiveness of a therapeutic agent targeting GLP1R for the treatment of a metabolic disorder, comprising: administering the therapeutic agent targeting GLP1R to the animal as described herein, and determining the effects of the therapeutic agent targeting GLP1R to the metabolism of the animal.

In some embodiments, the animal comprises one or more cells that express a GLP1R ligand. In some embodiments, animal is on a high-fat diet to induce obesity and high blood sugar. In some embodiments, determining the effects of the therapeutic agent targeting GLP1R to the metabolism of the animal comprises measuring the blood glucose of the animal. In some embodiments, determining the effects of the therapeutic agent targeting GLP1R to the metabolism of the animal comprises measuring the body weight of the animal.

In one aspect, the disclosure provides a method of determining effectiveness of a therapeutic agent targeting GLP1R and an additional therapeutic agent for the treatment of a metabolic disorder, comprising administering the therapeutic agent targeting GLP1R and the additional therapeutic agent to the animal as described herein; and determining the effects of the therapeutic agent targeting GLP1R to the metabolism of the animal. In some embodiments, the animal comprises one or more cells that express a GLP1R ligand. In some embodiments, the animal is on a high-fat diet to induce obesity and high blood sugar. In some embodiments, determining the effects of the therapeutic agent targeting GLP1R to the metabolism of the animal comprises measuring the blood glucose of the animal. In some embodiments, determining the effects of the therapeutic agent targeting GLP1R to the metabolism of the animal comprises measuring the body weight of the animal.

In one aspect, the disclosure provides a protein comprising an amino acid sequence, wherein the amino acid sequence comprises or consists of one of the following:
(a) an amino acid sequence set forth in SEQ ID NO: 2;
(b) an amino acid sequence that is at least 90% identical to SEQ ID NO: 2;
(c) an amino acid sequence that is at least 91%, 92%, 93%, 94%, 95%, 96%, 97%, 98%, or 99% identical to SEQ ID NO: 2;
(d) an amino acid sequence that is different from the amino acid sequence set forth in SEQ ID NO: 2 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2.

In one aspect, the disclosure provides a nucleic acid comprising a nucleotide sequence, wherein the nucleotide sequence comprises or consists of one of the following:
(a) a sequence that encodes the protein as described herein;
(b) SEQ ID NO: 3
(c) SEQ ID NO: 4
(d) SEQ ID NO: 5;
(e) SEQ ID NO: 6;
(f) SEQ ID NO: 7;
(g) SEQ ID NO: 8;
(h) SEQ ID NO: 9;
(i) SEQ ID NO: 10;
(j) a sequence that is at least 90% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10;
(k) a sequence that is at least 91%, 92%, 93%, 94%, 95%, 96%, 97%, 98%, or 99% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In one aspect, the disclosure provides a cell comprising the protein as described herein and/or the nucleic acid as described herein. In one aspect, the disclosure provides an animal comprising the protein as described herein and/or the nucleic acid as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram of the comparison between (1) mouse GL gene and (2) human GLP1R gene locus (not to scale). mExon4-5 are mExon4 and mExon5; mExon8-11 are mExon8, mExon9, mExon10 and mExon11; hExon2-3 are hExon2 and hExon3; hExon4-5 are hExon4 and hExon5; hExon8-11 are hExon8, hExon9, hExon10, and hExon11.

FIG. 2 is a schematic diagram of the humanization of mouse GLP1R gene (not to scale). chiExon4-5 are chiExon4 and chiExon5, chiExon8-11 are chiExon8, chiExon9, chiExon10, chiExon11.

FIG. 3 is a schematic diagram of GLP1R gene targeting strategy and targeting vector design (not to scale).

FIG. 4: Southern Blot results of GLP1R humanized mouse cells. WT is the wild-type control. 1-B02, 1-B11, 1-F08,-G11, 1-G12, 1-H05, 2-B01, 2-C01, and 2-H01 are the clone numbers.

FIGS. 5A-5D: GLP1R humanized 1 generation mouse tail gtype identification results. WT means wild type control; H₂O means water control; PC means positive control; F1-01, F1-02 are mouse numbers.

FIGS. 6A-6C: RT-PCR detection results in C57BL/6 mice and GLP1R gene humanized heterozygous mice. +/+ is C57BL/6 wild-type mouse; H/+ is GLP1R gene humanized heterozygote mouse; H₂O is the water control; and GAPDH is the glyceraldehyde-3-phosphate dehydrogenase internal control.

FIGS. 7A-7C: RT-PCR detection results in C57BL/6 mice and GLP1R humanized homozygous mice. +/+ is C57BL/6 wild-type mouse; H/H is GLP1R gene humanized homozygote mouse; H₂O is the water control; and GAPDHs the glyceraldehyde-3-phosphate dehydrogenase internal control.

FIG. 8: GLP1R protein detection results in C57BL/6 mice and GLP1R humaned homozygous mice. +/+ is C57BL/6 wild-type mouse; H/H is GLP1R humanized homozygous mouse; B-actin is β-actin internal reference.

FIGS. 9A-9I: IHC staining results of pancreas tissue of C57BL/6 mice and GLP1R humanized homozygous mice. +/+ is C57BL/6 mouse; H/H is GLP1R humanized homozygous mouse; ISO is isotype control.

FIG. 10: Drug efficacy experiment design (including e.g., grouping and administration plans).

FIG. 11: Changes in body weight of mice in each group during the experiment.

FIG. 12: Changes in non-fasting blood glucose of mice in each group during the experiment.

FIG. 13: Changes in fasting blood glucose of mice in each group during the experiment.

FIG. 14: Changes in IPGTT (glucose tolerance test) blood glucose concentration.

FIG. 15: Area under the curve (AUC) of IPGTT.

FIG. 16: The percentage of white blood cell subtypes in the spleen of wild-type C57BL/6 mice (C57BL/6) and GLP1R humanized homozygous mice (GLP1R).

FIG. 17: Percentage of T cell subtypes in the spleen of wild-type C57BL/6 mice and GLP1R humanized homozygous mice.

FIG. 18: Percentage of white blood cell subtypes in lymph nodes of wild-type C57BL/6 mice and GLP1R humanized homozygous mice.

FIG. 19: Percentage of T cell subtypes in lymph nodes of wild-type C57BL/6 mice and GLP1R humanized homozygous mice.

FIG. 20: Percentage of white blood cell subtypes in the peripheral blood of wild-type C57BL/6 mice and GLP1R humanized homozygous mice.

FIG. 21 Percentage of T cell subtypes in the peripheral blood of the spleen of wild-type C57BL/6 mice and GLP1R humanized homozygous mice.

FIG. 22: Efficacy experiment protocol and testing schedule.

FIG. 23: Changes in body weight of mice in each group during the experiment.

FIG. 24: Food intake of mice in each group 24 hours before the first administration and 24 hours after the first administration.

FIG. 25: Changes in non-fasting blood glucose of mice in each group during the experiment.

FIG. 26: Changes in IPGTT (glucose tolerance test) blood glucose concentration.

FIG. 27: Serum insulin of mice in each group during the experiment.

FIG. 28: Serum glucagon of mice in each group during the experiment.

FIG. 29: Serum GLP-1 of mice in each group during the experiment.

FIG. 30: Alignment between mouse GLP1R amino acid sequence (NP_067307.2; SEQ ID NO: 1) and human GLP1R amino acid sequence (NP_002053.3; SEQ ID NO: 2).

### DETAILED DESCRIPTION

This disclosure relates to transgenic non-human animal with human or chimeric (e.g., humanized) GLP1R (Glucagon-like peptide-1 receptor), and methods of use thereof.

GLP1R (Glucagon-like peptide-1 receptor) is a member of the B-type GPCR (G protein-coupled receptor) family, which is widely distributed in the islets, stomach, the small intestine, heart, kidney, brain and other tissues. Upon the binding of GLP-1 (Glucagon-like peptide-1, glucagon-like peptide-1), GLP1R activates downstream signaling pathways such as PKA, PI3K, MAPK, and participates in the release of insulin, R Cell proliferation, decreased glucagon, delayed gastric emptying, enhanced memory and other important physiological processes. In pancreatic islet cells, GLP1R mainly plays the role of promoting insulin release, increasing islet R cell regeneration, inhibiting R cell apoptosis, and reducing glucagon release. In gastrointestinal tissues, it can inhibit gastrointestinal peristalsis and secretion of gastric juice, delay gastric emptying, increase satiety. In the brain nerve tissue, it can protect nerve cells, reduce their apoptosis, enhance learning and memory, and control food intake to reduce weight. It is an internationally recognized treatment target for type II diabetes. There are already a number of peptide drugs targeting this receptor on the market including Exenatide (trade name Byetta/Bydureon), Liraglutide (trade name Victoza), Lixisenatide (trade name Lyxumia), Albiglutide (trade name Tanzeu), Dulaglutide (trade name Trulicity), etc. The annual sales of these drugs total more than tens of billions of dollars. In addition, recent studies have found that GLP1R is abnormally expressed in some tumor cells, such as insulinoma and thyroid papilloma. Other studies have found that GLP1R agonists can significantly inhibit the activation of human hepatic stellate cells in vitro and significantly improve the degree of cholestatic liver fibrosis in rats induced by common bile duct ligation, showing promise in preventing or treating non-alcoholic fatty liver disease, hyperlipidemia, arteriosclerosis and other diseases.

Experimental animal models are an indispensable research tool for studying the effects of GLP1R drugs. Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. However, due to the differences in physiology and pathology between animals and humans, coupled with the complexity of genes, how to construct an “effective” humanized animal model that approximates the physiological characteristics of humans is still one of the biggest challenges for new drug research and development.

The present disclosure provides a genetically modified animal expressing human or chimeric (e.g., humanized) GLP1R, and methods of use thereof and demonstrates that the genetically modified animals as described herein can be properly used in drug screening. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullisetal U. S. Pat. No. 4, 683, 195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wuetal. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### GLP1R

The GLP-1R is a 463-amino-acid transmembrane-spanning protein belonging to the family B/secretin GPCRs, mediating the effects of both endogenous GLP-1 peptides [four forms: GLP-1 (1-36)NH2, GLP-1 (7-36)NH2, GLP-1 (1-37) and GLP-1 (7-37)], as well as the endogenous peptide oxyntomodulin and exogenous peptide exendin-4. Characteristic of family B GPCRs, the GLP-1R possesses a long extracellular N-terminus with an α-helical region, five 0-strands forming two antiparallel β-sheets and six conserved cysteine residues that form disulfide interactions. Together, these features allow the receptor to adopt the classic ‘Sushi domain’ or ‘short consensus repeat’, which aids N-terminal stability and confers a high level of structural homology within the N-terminal regions of family B GPCRs. The large extracellular N-terminus has a significant role in peptide binding, supported by GLP-1 binding the isolated N-terminus of the GLP-1R and crystal structures of the isolated GLP-1R N-terminus in complex with GLP-1 and exendin peptides. Specifically, the C-terminus of the peptide interacts with the N-terminus of the receptor, which is proposed to be responsible for ligand recognition and specificity, while the N-terminus of the peptide is proposed to associate with the core of the receptor, and is suggested to have a major influence in signaling specificity and transmission.

The physiological changes observed with increases in GLP-1, including increases in insulin secretion and β-cell mass, rely on signaling via GLP-1R-mediated intracellular pathways. The GLP-1R is a pleiotropically coupled receptor, with evidence for signaling via multiple G-protein-coupled pathways including Gαs, Gαi, Gαo and Gαq/11. The GLP-1R is most well documented for its role in Gαs coupling, favouring production of cAMP through increasing enzymatic activity of adenylate cyclase. This subsequently promotes increases in both PKA (protein kinase A) and Epac2 (exchange protein activated by cAMP-2), which is directly involved in enhancing proinsulin gene transcription. Furthermore, GLP-1R activation induces membrane depolarization of β-cells through inhibition of K+ channels, allowing VDCCs (voltage-dependent Ca2+ channels) to open and acceleration of Ca2+ influx to occur, resulting in the exocytosis of insulin from 0-cells. Therefore the production of cAMP and influx of Ca2+ are vital components in the biosynthesis and secretion of insulin. GLP-1R activity also promotes EGFR (epidermal growth factor receptor) transactivation, PI3K (phosphoinositide β-kinase) activity, IRS-2 (insulin receptor substrate-2) signalling, and subsequently, ERK1/2 (extracellular-signalregulated kinase 1 and 2) activity, as well as nuclear translocation of PKCζ to mediate (β-cell proliferation and differentiation as well as promote insulin gene transcription.

A detailed description of GLP1R, and the use of anti-GLP1R antibodies to treat diseases are described, e.g., in Chunxia Liu, et al. (2020) GLP-1R agonists for the treatment of obesity: a patent review (2015-present), Expert Opinion on Therapeutic Patents, 30: 10, 781-794; Koole C, et al. Recent advances in understanding GLP-1R (glucagon-like peptide-1 receptor) function. Biochem Soc Trans. 2013 Feb. 1; 41 (1): 172-9; Heppner K M, et al. (2015) GLP-1 based therapeutics: simultaneously combating T2DM and obesity. Front. Neurosci. 9: 92; Bazarsuren et al., In vitro folding, functional characterization, and disulfide pattern of the extracellular domain of human GLP-1 receptor, Biophysical chemistry 96.2-3 (2002): 305-318; each of which is incorporated by reference in its entirety.

In human genomes, GLP1R gene (Gene ID: 2740) locus has 13 exons, exon 1, exon 2, exon 3, exon 4, and exon 5, exon 6, exon 7, exon 8, exon 9, exon 10, exon 11, exon 12 and exon 13. The GLP1R protein also has extracellular regions, transmembrane regions, and cytoplasmic regions. The nucleotide sequence for human GLP1R mRNA is NM_002062.5 (SEQ ID NO: 31), and the amino acid sequence for human GLP1R is NP_002053.3 (SEQ ID NO: 2). The location for each exon and each region in human GLP1R nucleotide sequence and amino acid sequence is listed below:

TABLE 1

| Human GLP1R | NM_002062.5 | NP_002053.3 |
|---|---|---|
| (approximate | 6682 bp | 463 aa |
| location) | SEQ ID NO: 31 | SEQ ID NO: 2 |
| Exon 1 | 1-138 | 1-26 |
| Exon 2 | 139-235 | 27-58 |
| Exon 3 | 236-343 | 59-94 |
| Exon 4 | 344-462 | 95-134 |
| Exon 5 | 463-569 | 135-170 |
| Exon 6 | 570-723 | 171-221 |
| Exon 7 | 724-883 | 222-274 |
| Exon 8 | 884-944 | 275-295 |
| Exon 9 | 945-1014 | 296-318 |
| Exon 10 | 1015-1103 | 319-348 |
| Exon 11 | 1104-1242 | 349-394 |
| Exon 12 | 1243-1284 | 395-408 |
| Exon 13 | 1285-6682 | 409-463 |
| Signal peptide | 61-129 | 1-23 |
| Chain | 130-1449 | 24-463 |
| Donor region in Example | 61-1452 | 1-463 |

The human GLP1R gene (Gene ID: 2740) is located in Chromosome 6 of the human genome, which is located from 39048781 to 39091303 of NC_000006.12 (GRCh38.p13 (GCF_000001405.39)). The 5′-UTR is from 39,048,781 to 39,048,840, exon 1 is from 39,048,781 to 39,048,918, the first intron is from 39,048,919 to 39,056,396, exon 2 is from 39,056,397 to 39,056,493, the second intron is from 39,056,494 to 39,057,471, exon 3 is from 39,057,472 to 39,057,579, the third intron is from 39,057,580 to 39,065,710, exon 4 is from 39,065,711 to 39,065,829, the forth intron is from 39,065,830 to 39,066,196, exon 5 is from 39,066,197 to 39,066,303, the fifth intron is from 39,066,304 to 39,072,861, exon 6 is from 39,072,862 to 39,073,015, the sixth intron is from 39,073,016 to 39,073,609, exon 7 is from 39,073,610 to 39,073,769, the seventh intron is from 39,073,770 to 39,078,321, exon 8 is from 39,078,322 to 39,078,382, the eighth intron is from 39,078,383 to 39,078,956, exon 9 is from 39,078,957 to 39,079,026, the ninth intron is from 39,079,027 to 39,079,111, exon 10 is from 39,079,112 to 39,079,200, the tenth intron is from 39,079,201 to 39,079,563, exon 11 is from 39,079,564 to 39,079,702, the 11th intron is from 39,079,703 to 39,080,697, exon 12 is from 39,080,698 to 39,080,739, the 12th intron is from 39,080,740 to 39,085,905, exon 13 is from 39,085,906 to 39,091,303, the 3′-UTR is from 39,086,074 to 39,091,303, based on transcript NM_002062.5. All relevant information for human GLP1R locus can be found in the NCBI website with Gene ID: 2740, which is incorporated by reference herein in its entirety.

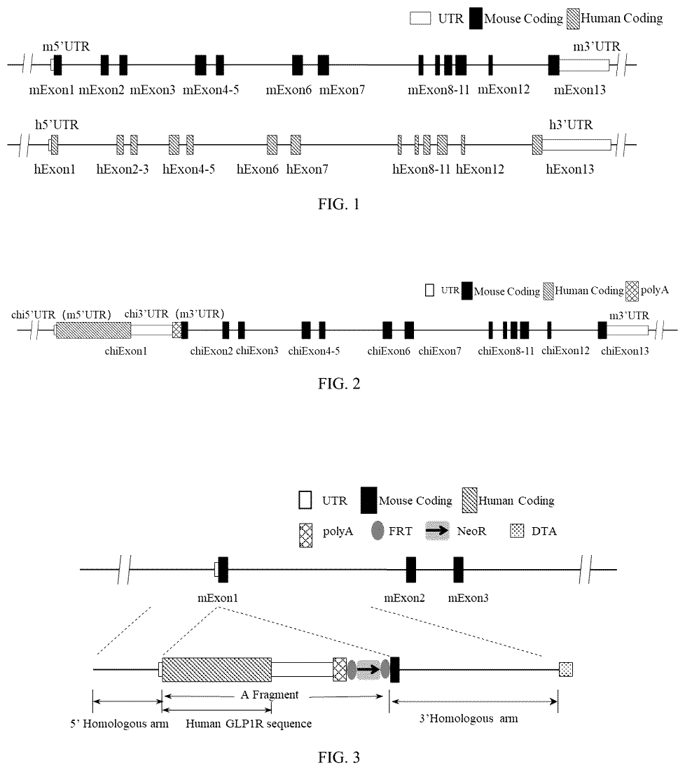

In mice, GLP1R gene locus has 13 exons, exon 1, exon 2, exon 3, exon 4, and exon 5, exon 6, exon 7, exon 8, exon 9, exon 10, exon 11, exon 12 and exon 13 (FIG. 1). The mouse GLP1R protein also has extracellular regions, transmembrane regions, and cytoplasmic regions. The nucleotide sequence for mouse GLP1R mRNA is NM_021332.2 (SEQ ID NO: 32), the amino acid sequence for mouse GLP1R is NP_067307.2 (SEQ ID NO: 1). The location for each exon and each region in the mouse GLP1R nucleotide sequence and amino acid sequence is listed below:

TABLE 2

| Mouse Glp1r | NM_021332.2 | NP_067307.2 |
|---|---|---|
| (approximate | 1480 bp | 463aa |
| location) | SEQ ID NO: 32 | SEQ ID NO: 1 |
| Exon 1 | 1-88 | 1-26 |
| Exon 2 | 89-185 | 27-58 |
| Exon 3 | 186-293 | 59-94 |
| Exon 4 | 294-412 | 95-134 |
| Exon 5 | 413-519 | 135-170 |
| Exon 6 | 520-673 | 171-221 |
| Exon 7 | 674-833 | 222-274 |
| Exon 8 | 834-894 | 275-295 |
| Exon 9 | 895-964 | 296-318 |
| Exon 10 | 965-1053 | 319-348 |
| Exon 11 | 1054-1192 | 349-394 |
| Exon 12 | 1193-1234 | 395-408 |
| Exon 13 | 1235-1480 | 409-463 |
| Signal peptide | 11-73 | 1-21 |
| Chain | 74-1399 | 22-463 |
| | | (GPCR, crossing |
| | | membrane 8 times) |
| Insert site in | between 10 and 11 | — |
| Example | | |

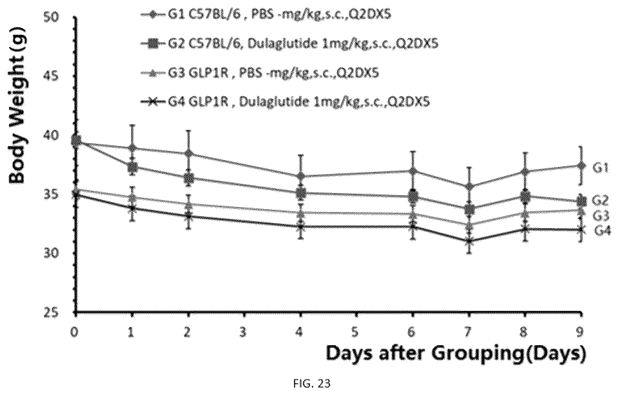

The mouse Glp1r gene (Gene ID: 14652) is located in Chromosome 17 of the mouse genome, which is located from 30901867 to 30936510 of NC_000083.6 (GRCm38.p6 (GCF_000001635.26)). The 5′-UTR is from 30,901,817 to 30,901,876, exon 1 is from 30,901,817 to 30,901,954, the first intron is from 30,901,955 to 30,908,120, exon 2 is from 30,908,121 to 30,908,217, the second intron is from 30,908,218 to 30,909,223, exon 3 is from 30,909,224 to 30,909,331, the third intron is from 30,909,332 to 30,918,879, exon 4 is from 30,918,880 to 30,918,998, the forth intron is from 30,918,999 to 30,919,355, exon 5 is from 30,919,356 to 30,919,462, the fifth intron is from 30,919,463 to 30,924,500, exon 6 is from 30,924,501 to 30,924,654, the sixth intron is from 30,924,655 to 30,925,517, exon 7 is from 30,925,518 to 30,925,677, the seventh intron is from 30,925,678 to 30,929,936, exon 8 is from 30,929,937 to 30,929,997, the eighth intron is from 30,929,998 to 30,930,526, exon 9 is from 30,930,527 to 30,930,596, the ninth intron is from 30,930,597 to 30,930,717, exon 10 is from 30,930,718 to 30,930,806, the tenth intron is from 30,930,807 to 30,931,148, exon 11 is from 30,931,149 to 30,931,287, the 11th intron is from 30,931,288 to 30,932,612, exon 12 is from 30,932,613 to 30,932,654, the 12th intron is from 30,932,655 to 30,936,264, exon 13 is from 30,936,265 to 30,940,791, the 3′-UTR is from 30,936,433 to 30,940,791, based on transcript NM_021332.2. All relevant information for mouse Glp1r locus can be found in the NCBI website with Gene ID: 14652, which is incorporated by reference herein in its entirety. FIG. 23 shows the alignment between mouse GLP1R gene and human GLP1R gene locus.

GLP1R genes, proteins, and locus of the other species are also known in the art. For example, the gene ID for GLP1R in Rattus norvegicus is 25051, the gene ID for GLP1R in Macaca mulatta (Rhesus monkey) is 719548, the gene ID for GLP1R in Canis lupusfamiliaris (dog) is 481778, and the gene ID for GLP1R in Bos taurus (cattle) is 517420. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) GLP1R nucleotide sequence and/or amino acid sequences. In some embodiments, the entire sequence of mouse exon 1, exon 2, exon 3, exon 4, exon 5, exon 6, exon 7, exon 8, exon 9, exon 10, exon 11, exon 12, exon 13, signal peptide, extracellular regions (e.g., 1^{st}, 2^{nd}, 3^{nd}, 4^{th} extracellular region from the N terminal), transmembrane region, and/or cytoplasmic region (e.g., 1^{st}, 2^{nd}, 3^{rd}, 4^{th} cytoplasmic region from the N terminal) are replaced by the corresponding human sequence. In some embodiments, a “region” or “portion” of mouse exon 1, exon 2, exon 3, exon 4, exon 5, exon 6, exon 7, exon 8, exon 9, exon 10, exon 11, exon 12, exon 13, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, exon 10, exon 11, exon 12, exon 13, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, exon 8, exon 9, exon 10, exon 11, exon 12, and/or exon 13 are replaced by the human exon 1, exon 2, exon 3, exon 4, exon 5, exon 6, exon 7, exon 8, exon 9, exon 10, exon 11, exon 12, and/or exon 13 sequence.

In some embodiments, the present disclosure also provides a chimeric (e.g., humanized) GLP1R nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse GLP1R gene, mouse GLP1R amino acid sequence (e.g., SEQ ID NO: 1), or a portion thereof (e.g., exon 1, exon 2, exon 3, exon 4, exon 5, exon 6, exon 7, exon 8, exon 9, exon 10, exon 11, exon 12, and exon 13); and in some embodiments, at least 1%, 2%, 3%, 4%, 5%, 6%, 7%, 8%, 9%, 10%, 15%, 20%, 25%, 30%, 35%0, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human GLP1R gene sequence, human GLP1R amino acid sequence (e.g., SEQ ID NO: 2), or a portion thereof (e.g., exon 1, exon 2, exon 3, exon 4, exon 5, exon 6, exon 7, exon 8, exon 9, exon 10, exon 11, exon 12, and exon 13).

In some embodiments, the nucleic acids as described herein are operably linked to a promotor or regulatory element, e.g., an endogenous mouse GLP1R promotor, an inducible promoter, an enhancer, and/or mouse or human regulatory elements.

In some embodiments, the nucleic acids as described herein are operably linked to a Woodchuck Hepatitis Virus (WHP) Posttranscriptional Regulatory Element (WPRE). In some embodiments, the nucleic acids as described herein are operably linked to a polyA (polyadenylation) signal sequence.

In some embodiments, the polyA (polyadenylation) signal sequence has a sequence that is at least 70%, 80%, 90%, or 95% identical to SEQ ID NO: 7.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse GLP1R nucleotide sequence (e.g., exon 1, exon 2, exon 3, exon 4, exon 5, exon 6, exon 7, exon 8, exon 9, exon 10, exon 11, exon 12, and/or exon 13).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse GLP1R nucleotide sequence (e.g., exon 1, exon 2, exon 3, exon 4, exon 5, exon 6, exon 7, exon 8, exon 9, exon 10, exon 11, exon 12, exon 13).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human GLP1R nucleotide sequence (e.g., exon 1, exon 2, exon 3, exon 4, exon 5, exon 6, exon 7, exon 8, exon 9, exon 10, exon 11, exon 12, exon 13).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human GLP1R nucleotide sequence (e.g., exon 1, exon 2, exon 3, exon 4, exon 5, exon 6, exon 7, exon 8, exon 9, exon 10, exon 11, exon 12, exon 13).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse GLP1R amino acid sequence (e.g., exon 1, exon 2, exon 3, exon 4, exon 5, exon 6, exon 7, exon 8, exon 9, exon 10, exon 11, exon 12, exon 13 or SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse GLP1R amino acid sequence (e.g., exon 1, exon 2, exon 3, exon 4, exon 5, exon 6, exon 7, exon 8, exon 9, exon 10, exon 11, exon 12, exon 13 or SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human GLP1R amino acid sequence (e.g., exon 1, exon 2, exon 3, exon 4, exon 5, exon 6, exon 7, exon 8, exon 9, exon 10, exon 11, exon 12, exon 13 or SEQ ID NO: 2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human GLP1R amino acid sequence (e.g., exon 1, exon 2, exon 3, exon 4, exon 5, exon 6, exon 7, exon 8, exon 9, exon 10, exon 11, exon 12, exon 13 or SEQ ID NO: 2).

The present disclosure also provides a humanized GLP1R mouse amino acid sequence, wherein the amino acid sequence is selected from the group consisting of:
a) an amino acid sequence shown in SEQ ID NO: 2;
b) an amino acid sequence having a homology of at least 90% with or at least 90% identical to the amino acid sequence shown in SEQ ID NO: 2;
c) an amino acid sequence encoded by a nucleic acid sequence, wherein the nucleic acid sequence is able to hybridize to a nucleotide sequence encoding the amino acid shown in SEQ ID NO: 2 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2;
e) an amino acid sequence that is different from the amino acid sequence shown in SEQ ID NO: 2 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 2.

The present disclosure also relates to a GLP1R nucleic acid (e.g., DNA or RNA) sequence, wherein the nucleic acid sequence can be selected from the group consisting of:
a) a nucleic acid sequence as shown in SEQ ID NO: 5, or a nucleic acid sequence encoding a homologous GLP1R amino acid sequence of a humanized mouse;
b) a nucleic acid sequence that is shown in SEQ ID NO: 5;
c) a nucleic acid sequence that is able to hybridize to the nucleotide sequence as shown in SEQ ID NO: 5 under a low stringency condition or a strict stringency condition;
d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 5;
e) a nucleic acid sequence that encodes an amino acid sequence, wherein the amino acid sequence has a homology of at least 90% with or at least 90% identical to the amino acid sequence shown in SEQ ID NO: 2;
f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 2;
g) a nucleic acid sequence that encodes an amino acid sequence, wherein the amino acid sequence is different from the amino acid sequence shown in SEQ ID NO: 2 by no more than 10, 9, 8, 7, 6, 5, 4, 3, 2 or no more than 1 amino acid; and/or
h) a nucleic acid sequence that encodes an amino acid sequence, wherein the amino acid sequence comprises a substitution, a deletion and/or insertion of one or more amino acids to the amino acid sequence shown in SEQ ID NO: 2.

The present disclosure further relates to a GLP1R genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 5.

The disclosure also provides an amino acid sequence that has a homology of at least 90% with, or at least 90% identical to the sequence shown in SEQ ID NO: 2, and has protein activity. In some embodiments, the homology with the sequence shown in SEQ ID NO: 2 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 2 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 5, and encodes a polypeptide that has protein activity. In some embodiments, the homology with the sequence shown in SEQ ID NO: 5 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) GLP1R from an endogenous non-human GLP1R locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous GLP1R locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wildtype amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized GLP1R gene or a humanized GLP1R nucleic acid. In some embodiments, at least one or more portions of the gene or the nucleic acid is from the human GLP1R gene, at least one or more portions of the gene or the nucleic acid is from a non-human GLP1R gene. In some embodiments, the gene or the nucleic acid comprises a sequence that encodes a GLP1R protein. The encoded GLP1R protein is functional or has at least one activity of the human GLP1R protein or the non-human GLP1R protein, e.g., binding with human or non-human GLP1.

In some embodiments, the chimeric protein or the chimeric polypeptide is a humanized GLP1R protein or a humanized GLP1R polypeptide. In some embodiments, at least one or more portions of the amino acid sequence of the protein or the polypeptide is from a human GLP1R protein, and at least one or more portions of the amino acid sequence of the protein or the polypeptide is from a non-human GLP1R protein. The humanized GLP1R protein or the humanized GLP1R polypeptide is functional or has at least one activity of the human GLP1R protein or the non-human GLP1R protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea orMuroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 12951/SV, 12951/SvIm), 12952, 12954, 12955, 12959/SvEvH, 12956 (129/SvEvTac), 12957, 12958, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized GLP1R animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2RT knockout mice, NOD/SCID/γcnull mice (Ito, M. et al., NOD/SCID/γcnull mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human GLP1R locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Ry knockout mice, NOD/SCID/yc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature GLP1R coding sequence with human mature GLP1R coding sequence or an insertion of human mature GLP1R coding sequence or chimeric GLP1R coding sequence.

Genetically modified non-human animals that comprise a modification of an endogenous non-human GLP1R locus. In some embodiments, the modification can comprise a human nucleic acid sequence encoding at least a portion of a mature GLP1R protein (e.g., at least 10%, 20%, 30%, 40%, 50%, 60%, 70%, 80%, 90%, 95%, 96%, 97%, 98%, or 99% identical to the mature GLP1R protein sequence). Although genetically modified cells are also provided that can comprise the modifications described herein (e.g., ES cells, somatic cells), in many embodiments, the genetically modified non-human animals comprise the modification of the endogenous GLP1R locus in the germline of the animal.

Genetically modified animals can express a human GLP1R and/or a chimeric (e.g., humanized) GLP1R from endogenous mouse loci, wherein the endogenous mouse GLP1R gene has been replaced with a human GLP1R gene and/or a nucleotide sequence that encodes a region of human GLP1R sequence or an amino acid sequence that is at least 10%, 20%, 30%, 40%, 50%, 60%, 70%, 80%, 90%, 95%, 96%, 97%, 98%, or 99% identical to the human GLP1R sequence. In various embodiments, an endogenous non-human GLP1R locus is modified in whole or in part to comprise human nucleic acid sequence encoding at least one protein-coding sequence of a mature GLP1R protein.

In some embodiments, the genetically modified mice express the human GLP1R and/or chimeric GLP1R (e.g., humanized GLP1R) from endogenous loci that are under control of mouse promoters and/or mouse regulatory elements. The replacement (s) at the endogenous mouse loci provide non-human animals that express human GLP1R or chimeric GLP1R (e.g., humanized GLP1R) in appropriate cell types and in a manner that does not result in the potential pathologies observed in some other transgenic mice known in the art. The human GLP1R or the chimeric GLP1R (e.g., humanized GLP1R) expressed in animal can maintain one or more functions of the wildtype mouse or human GLP1R in the animal. For example, human or non-human GLP1R ligands (e.g., GLP1) can bind to the expressed GLP1R, activates adenylyl cyclase pathway, e.g., activates adenylyl cyclase pathway by at least 1 fold, 2 folds, 5 folds, or 10 folds. Furthermore, in some embodiments, the animal does not express endogenous GLP1R. As used herein, the term “endogenous GLP1R” refers to GLP1R protein that is expressed from an endogenous GLP1R nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human GLP1R (SEQ ID NO: 2). In some embodiments, the genome comprises a sequence encoding an amino acid sequence that is at least 70%, 75%, 80%, 85%, 90%, 95%, 99%, or 100% identical to SEQ ID NO: 2.

The genome of the genetically modified animal can comprise a replacement at an endogenous GLP1R gene locus of a sequence encoding a region of endogenous GLP1R with a sequence encoding a corresponding region of human GLP1R. In some embodiments, the sequence that is replaced is any sequence within the endogenous GLP1R gene locus, e.g., exon 1, exon 2, exon 3, exon 4, exon 5, exon 6, exon 7, exon 8, exon 9, exon 10, exon 11, exon 12, exon 13, 5′-UTR, 3′-UTR, the first intron, the second intron, the third intron, and the fourth intron, the 1st, the 2nd, the 3rd, the 4th extracellular region from the N terminal, the 1st, the 2nd, the 3rd, the 4th cytoplasmic region, etc. In some embodiments, the sequence that is replaced is within the exon 1 of the endogenous GLP1R gene.

In some embodiments, a sequence that encodes an amino acid sequence (e.g., human GLP1R or chimeric GLP1R) is inserted after 5′-UTR (e.g., immediately after 5′-URT), or immediately before the start codon (e.g., within 10, 20, 30, 40, 50, 60, 70, 80, 90, or 100 nucleic acids). The start codon is the first codon of a messenger RNA (mRNA) transcript translated by a ribosome. The start codon always codes for methionine in eukaryotes and a modified Met (fMet) in prokaryotes. The most common start codon is ATG (or AUG in mRNA).

In some embodiments, the inserted sequence further comprises a stop codon (e.g., TAG, TAA, TGA). The stop codon (or termination codon) is a nucleotide triplet within messenger RNA that signals a termination of translation into proteins. Thus, the endogenous sequence after the stop codon will not be translated into proteins. In some embodiments, at least one exons of (e.g., exon 1, exon 2, exon 3, exon 4, exon 5, exon 6, exon 7, exon 8, exon 9, exon 10, exon 11, exon 12, and/or exon 13) of the endogenous GLP1R gene are not translated into proteins.

The genetically modified animal can have one or more cells expressing a human or chimeric GLP1R (e.g., humanized GLP1R) having one or more extracellular regions and one or more cytoplasmic regions, wherein one of the extracellular regions comprises a sequence that is at least 50%, 60%, 70%, 80%, 90%, 95%, 99% identical to one of the extracellular regions of human GLP1R. In some embodiments, one of the extracellular regions of the humanized GLP1R has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to one of the extracellular regions of human GLP1R. Because human GLP1R and non-human GLP1R (e.g., mouse GLP1R) sequences, in many cases, are different, antibodies that bind to human GLP1R will not necessarily have the same binding affinity with non-human GLP1R or have the same effects to non-human GLP1R. Therefore, the genetically modified animal having a human or a humanized extracellular region can be used to better evaluate the effects of anti-human GLP1R antibodies in an animal model. In some embodiments, the genome of the genetically modified animal comprises a sequence encoding an amino acid sequence that corresponds to part or the entire sequence of exon 1, exon 2, exon 3, exon 4, exon 5, exon 6, exon 7, exon 8, exon 9, exon 10, exon 11, exon 12, and/or exon 13 of human GLP1R, or part or the entire sequence of one or more extracellular regions of human GLP1R (with or without signal peptide).

In some embodiments, the non-human animal can have, at an endogenous GLP1R gene locus, a nucleotide sequence encoding a chimeric human/non-human GLP1R polypeptide, wherein a human portion of the chimeric human/non-human GLP1R polypeptide comprises a portion of one or more human GLP1R extracellular regions, and wherein the animal expresses a functional GLP1R on a surface of a cell of the animal. The human portion of the chimeric human/non-human GLP1R polypeptide can comprise a portion of exon 1, exon 2, exon 3, exon 4, exon 5, exon 6, exon 7, exon 8, exon 9, exon 10, exon 11, exon 12, and/or exon 13 of human GLP1R. In some embodiments, the human portion of the chimeric human/non-human GLP1R polypeptide can comprise a sequence that is at least 80%, 85%, 90%, 95%, or 99% identical to SEQ ID NO: 2.

In some embodiments, the non-human animal genome also includes other genetic modifications. In some embodiments, the other genes include one of human PD-1, PD-L1, CTLA4, LAG3, IL4, IL6, or CCR4 genes, or a combination of two or more.

In some embodiments, the nucleotide sequence of the humanized GLP1R gene includes one of the following groups:
(1) all or part of the nucleotide sequence shown in SEQ ID NO: 5;
(2) at least 60%, 65%, 70%, 75%, 80%, 85%, 90%, 91%, 92%, 93% identity with the nucleotide sequence shown in SEQ ID NO: 5, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 5; or
(4) the nucleotide sequence shown in the nucleotide sequence shown in SEQ ID NO: 5, including substitution, deletion and/or insertion of one or more nucleotides.

In some embodiments, the humanized GLP1R gene further comprises an auxiliary sequence, which is connected after the human GLP1R gene. Further preferably, the auxiliary sequence is selected from a stop codon, a flip sequence or a knockout sequence. More preferably, the auxiliary sequence is 3′UTR and/or polyA of a non-human animal.

In some embodiments, the nucleotide sequence of the humanized GLP1R gene includes one of the following groups:
(1) all or part of the nucleotide sequence shown in SEQ ID NO: 6;
(2) at least 60%, 65%, 70%, 75%, 80%, 85%, 90%, 91%, 92%, 93% identity with the nucleotide sequence shown in SEQ ID NO: 6, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 6; or
(4) the nucleotide sequence shown in the nucleotide sequence shown in SEQ ID NO: 6, including substitution, deletion and/or insertion of one or more nucleotides.

In some embodiments, the nucleotide sequence of the humanized GLP1R gene includes one of the following groups:
(1) all or part of the nucleotide sequence shown in SEQ ID NO: 7;
(2) at least 60%, 65%, 70%, 75%, 80%, 85%, 90%, 91%, 92%, 93% identity with the nucleotide sequence shown in SEQ ID NO: 7, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 7; or
(4) the nucleotide sequence shown in the nucleotide sequence shown in SEQ ID NO: 7, including substitution, deletion and/or insertion of one or more nucleotides.

In some embodiments, the non-human animal can have transcribed mRNA sequence including one of the following groups:
(1) all or part of the nucleotide sequence shown in SEQ ID NO: 11;
(2) at least 60%, 65%, 70%, 75%, 80%, 85%, 90%, 91%, 92%, 93% identity with the nucleotide sequence shown in SEQ ID NO: 11, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 11; or
(4) the nucleotide sequence shown in the nucleotide sequence shown in SEQ ID NO: 11, including substitution, deletion and/or insertion of one or more nucleotides.

In some embodiments, the humanized GLP1R gene also includes a specific inducer or repressor. Further preferably, the specific inducer or repressor can be a conventional inducing or repressing substance.

In a specific embodiment of the present invention, the specific inducer is selected from the tetracycline system (Tet-Off System/Tet-On System) or the tamoxifen system (Tamoxifen System).

In some embodiments, the non-human portion of the chimeric human/non-human GLP1R polypeptide comprises transmembrane and/or cytoplasmic regions of an endogenous non-human GLP1R polypeptide. There may be several advantages that are associated with the transmembrane and/or cytoplasmic regions of an endogenous non-human GLP1R polypeptide. For example, once a GLP1R ligand (e.g., GLP1) or a therapeutic agent targeting GLP1R (e.g., an anti-GLP1R antibody) binds to GLP1R, they can properly transmit extracellular signals into the cells and initiate the downstream pathway.

Furthermore, the genetically modified animal can be heterozygous with respect to the replacement or insertion at the endogenous GLP1R locus, or homozygous with respect to the replacement or insertion at the endogenous GLP1R locus.

In some embodiments, the genetically modified animal (e.g., a rodent) comprises a humanization of an endogenous GLP1R gene, wherein the humanization comprises a replacement at the endogenous rodent GLP1R locus of a nucleic acid comprising an exon of a GLP1R gene with a nucleic acid sequence comprising at least one exon of a human GLP1R gene to form a modified GLP1R gene.

In some embodiments, the genetically modified animal (e.g., a rodent) comprises an insertion at the endogenous rodent GLP1R locus of a nucleic acid sequence comprising at least one exon of a human GLP1R gene to form a modified GLP1R gene.

In some embodiments, the expression of the modified GLP1R gene is under control of regulatory elements at the endogenous GLP1R locus. In some embodiments, the modified GLP1R gene is operably linked to a WPRE element.

In some embodiments, the humanized GLP1R locus lacks a human GLP1R 5′-UTR. In some embodiment, the humanized GLP1R locus comprises a rodent (e.g., mouse) 5′-UTR. In some embodiments, the humanization comprises a human 3′-UTR. In appropriate cases, it may be reasonable to presume that the mouse and human GLP1R genes appear to be similarly regulated based on the similarity of their 5′-flanking sequence. As shown in the present disclosure, humanized GLP1R mice that comprise an insertion at an endogenous mouse GLP1R locus, which retain mouse regulatory elements but comprise a humanization of GLP1R encoding sequence, do not exhibit obvious pathologies. Both genetically modified mice that are heterozygous or homozygous for humanized GLP1R are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized GLP1R gene.

The present disclosure further relates to a cell or cell line, or a primary cell culture thereof derived from the non-human mammal or an offspring thereof, and the tissue, organ or a culture thereof derived from the non-human mammal or an offspring thereof.

The present disclosure also provides non-human mammals produced by any of the methods described herein. In some embodiments, a non-human mammal is provided; and the genetically modified animal contains the DNA encoding human or humanized GLP1R in the genome of the animal.

In some embodiments, the non-human mammal comprises the genetic construct as described herein (e.g., gene construct as shown in FIG. 3). In some embodiments, a non-human mammal expressing human or humanized GLP1R is provided. In some embodiments, the tissue-specific expression of human or humanized GLP1R protein is provided.

In some embodiments, the expression of human or humanized GLP1R in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a rodent, e.g., a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human GLP1R protein or chimeric GLP1R protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized GLP1R protein.

### Vectors

The present disclosure relates to a targeting vector, comprising: a) a DNA fragment homologous to the 5′ end of a region to be altered (5′ arm), which is selected from the GLP1R gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the GLP1R gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000083.6; c) the DNA fragment homologous to the 3′ end of the region to be altered (3′ arm) is selected from the nucleotide sequences that have at least 90% homology to the NCBI accession number NC_000083.6.

In some embodiments, a) the DNA fragment homologous to the 5′ end of a region to be altered (5′ arm) is selected from the nucleotides from the position 30897870 to 30901876 of the NCBI accession number NC_000083.6; c) the DNA fragment homologous to the 3′ end of the region to be altered (3′ arm) is selected from the nucleotides from the position 30901880 to 30906647 of the NCBI accession number NC_000083.6.

In some embodiments, the length of the selected genomic nucleotide sequence in the targeting vector can be more than about 0.8 kb, about 1 kb, about 1.2 kb, or about 1.4 kb.

In some embodiments, the region to be altered is exon 1, exon 2, exon 3, exon 4, exon 5, exon 6, exon 7, exon 8, exon 9, exon 10, exon 11, exon 12, and/or exon 13 of GLP1R gene.

The targeting vector can further include a selection gene marker.

In some embodiments, the sequence of the 5′ arm is shown in SEQ ID NO: 3; and the sequence of the 3′ arm is shown in SEQ ID NO: 4.

In some embodiments, the sequence is derived from human. For example, the target region in the targeting vector is a part or entirety of the nucleotide sequence of a human GLP1R or a chimeric GLP1R. In some embodiments, the nucleotide sequence of the humanized GLP1R encodes the entire or the part of human GLP1R protein (SEQ ID NO: 2).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous GLP1R gene locus, a sequence encoding a region of an endogenous GLP1R with a sequence encoding a corresponding region of human GLP1R, a sequencing encoding human GLP1R, or a sequencing encoding chimeric GLP1R.

In some embodiments, the disclosure provides inserting in at least one cell of the animal, at an endogenous GLP1R gene locus, a sequence encoding a human GLP1R or a chimeric GLP1R.

In some embodiments, the genetic modification occurs in a germ cell, a somatic cell, a blastocyst, or a fibroblast, etc. The nucleus of a somatic cell or the fibroblast can be inserted into an enucleated oocyte.

FIG. 3 shows a humanization strategy for a mouse GLP1R locus. In FIG. 3, the targeting strategy involves a vector comprising the 5′ end homologous arm, human GLP1R gene fragment or chimeric GLP1R gene fragment, 3′ homologous arm. The process can involve inserting a human sequence by homologous recombination. In some embodiments, the cleavage at the upstream and the downstream of the target site (e.g., by zinc finger nucleases, TALEN or CRISPR) can result in DNA double strands break, and the homologous recombination is used to insert the human GLP1R sequence.

Thus, in some embodiments, the methods for making a genetically modified, humanized animal, can include the step of inserting at an endogenous GLP1R locus (or site), a sequence encoding a human GLP1R or a chimeric GLP1R. The sequence can include a region (e.g., a part or the entire region) of exon 1, exon 2, exon 3, exon 4, exon 5, exon 6, exon 7, exon 8, exon 9, exon 10, exon 11, exon 12, exon 13 of a human GLP1R gene. In some embodiments, the sequence includes a region of exon 1, exon 2, exon 3, exon 4, exon 5, exon 6, exon 7, exon 8, exon 9, exon 10, exon 11, exon 12, exon 13 of a human GLP1R gene (e.g., SEQ ID NO: 2). In some embodiments, the endogenous GLP1R locus is exon 1, exon 2, exon 3, exon 4, exon 5, exon 6, exon 7, exon 8, exon 9, exon 10, exon 11, exon 12, and/or exon 13 of mouse GLP1R (e.g., SEQ ID NO: 1). In some embodiments, the region is located within exon 1 of GLP1R.

In some embodiments, the methods of modifying a GLP1R locus of a mouse to express a chimeric human/mouse GLP1R peptide can include the steps of replacing at the endogenous mouse GLP1R locus a nucleotide sequence encoding a mouse GLP1R with a nucleotide sequence encoding a human GLP1R, thereby generating a sequence encoding a chimeric human/mouse GLP1R.

The present disclosure further provides a method for establishing a GLP1R gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

Methods of Using Genetically Modified Animals Insertion of human genes in a non-human animal at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized GLP1R protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized GLP1R, which are useful for testing agents that can decrease or block the interaction between GLP1R and GLP1R ligands (e.g., GLP1) or the interaction between GLP1R and anti-human GLP1R antibodies, testing whether an agent can increase or decrease the GLP1R pathway activity, and/or determining whether an agent is an GLP1R agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of a GLP1R targeting agent for the treatment of cancer (e.g., insulinoma and thyroid papilloma). The methods involve administering the agent (e.g., anti-human GLP1R antibody or anti-human GLP1 antibody) to the animal as described herein, wherein the animal has a tumor; and determining the inhibitory effects of the agent to the tumor. In some embodiments, the agent is an anti-human GLP1R antibody or anti-human GLP1 antibody.

In some embodiments, the genetically modified animals can be used for determining whether an agent (e.g., an anti-GLP1R antibody or a fusion protein) is a GLP1R agonist or antagonist. In some embodiments, the methods as described herein are also designed to determine the effects of the agent (e.g., anti-GLP1R antibodies) on GLP1R, e.g., whether the agent can change the glucose metabolism of the genetically modified animals. In some embodiments, the genetically modified animals can be used for determining the effective dosage of a therapeutic agent for treating a disease in the subject, e.g., metabolic disorders.

In some embodiments, the agent targeting GLP1R (e.g., an antibody) is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. In some embodiments, the tumor is insulinoma or thyroid papilloma.

In some embodiments, the agent is designed for treating various immune-related diseases. Thus, the methods as described herein can be used to determine the effectiveness of an agent targeting GLP1R (e.g., anti-GLP1R antibody) in treating the immune-related diseases. The immune-related diseases include but are not limited to allergies, asthma, dermatitis, myocarditis, nephritis, hepatitis, systemic lupus erythematosus, rheumatoid arthritis, scleroderma, hyperthyroidism, and primary thrombocytopenia Purpura, autoimmune hemolytic anemia, ulcerative colitis, autoimmune liver disease, diabetes, pain or neurological disorders, etc.

In some embodiments, the agent is designed for treating cardiovascular diseases. Thus, the methods as described herein can be used to determine the effectiveness of an agent targeting GLP1R (e.g., anti-GLP1R antibody) in treating cardiovascular diseases, e.g., hypertension, angina pectoris, myocardial infarction, coronary heart disease, heart failure, arrhythmia, endocarditis, pericarditis, non-alcoholic fatty liver disease, hyperlipidemia, arteriosclerosis, etc.

In some embodiments, the agent is designed for treating metabolic disorders. Thus, the methods as described herein can be used to determine the effectiveness of an agent targeting GLP1R (e.g., anti-GLP1R antibody) in treating metabolic disorders, e.g., diabetes, diabetic ketoacidosis, and hyperglycemia, hyperosmolar syndrome, hypoglycemia, gout, protein-energy malnutrition, vitamin A deficiency, scurvy or vitamin D deficiency. It can also be metabolic encephalopathy or congenital metabolic disorder.

In some embodiments, the methods as described herein can be used to evaluate the effects of an agent for promoting insulin release and/or islet p cell regeneration, inhibiting R cell apoptosis, and reducing glucagon release. In some embodiments, the methods as described herein can be used to evaluate treatment for metabolic disorders such as diabetes and obesity, and neurological diseases, such as Alzheimer's disease, dementia, Parkinson's disease, Parkinsonian Syndromes, etc.

The present disclosure also provides a method for screening a specific modulator of human GLP1R.

In some embodiments, the modulator is selected from CAR-T and drugs. Further preferably, the drug is an antibody or a small molecule drug.

In some embodiments, the modulator is a monoclonal antibody or a bispecific antibody or a combination of two or more drugs.

In some embodiments, the agent is a GLP1-Fc fusion protein.

In some embodiments, the detection includes determining the size and/or proliferation rate of tumor cells.

In some embodiments, the detection method includes vernier caliper measurement, flow cytometry detection and/or in vivo animal imaging detection.

In some embodiments, the detection includes assessing individual body weight, fat mass, activation pathway, neuroprotective activity, or metabolic changes, and the metabolic changes include changes in food consumption or water consumption.

In some embodiments, the tumor cells are derived from human or non-human animals.

In some embodiments, the screening method for the human GLP1R specific modulator is not a treatment method. This screening method is used to screen or evaluate drugs, test and compare the efficacy of candidate drugs to determine which candidate drugs can be used as drugs and which cannot be used as drugs, or to compare the sensitivity of different drugs, that is, the therapeutic effect is not inevitable and is just a possibility.

The present disclosure also provides methods of determining toxicity of an antibody (e.g., anti-GLP1R antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the GLP1R gene function, human GLP1R antibodies, drugs for human GLP1R targeting sites, the drugs or efficacies for human GLP1R targeting sites, the drugs for metabolic disorders.

Genetically Modified Animal Model with Two or More Human or Chimeric Genes

The present disclosure further relates to methods for generating genetically modified animal model with two or more human or chimeric genes. The animal can comprise a human or chimeric GLP1R gene and a sequence encoding an additional human or chimeric protein.

In some embodiments, the additional human or chimeric protein can be cytotoxic T-lymphocyte-associated protein 4 (CTLA-4), Lymphocyte Activating 3 (LAG-3), B And T Lymphocyte Associated (BTLA), Glucagon-like peptide-1 (GLP1), CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (TNFRSF4 or OX40).

In some embodiments, the animal has one or more cells expressing human or chimeric GLP1.

The methods of generating genetically modified animal model with two or more human or chimeric genes (e.g., humanized genes) can include the following steps:
(a) using the methods of introducing human GLP1R gene or chimeric GLP1R gene as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric CTLA-4, LAG-3, BTLA, GLP1, CD27, CD28, CD40, CD47, CD137, CD154, TIGIT, TIM-3, GITR, SIRPa, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the GLP1R humanization is directly performed on a genetically modified animal having a human or chimeric CTLA-4, BTLA, GLP1, CD27, CD28, CD40, CD47, CD137, CD154, TIGIT, TIM-3, GITR, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-GLP1R antibody and an additional therapeutic agent for the treatment of various disease. The methods include administering the anti-GLP1R antibody and the additional therapeutic agent to the animal; and determining the effects of the combined treatment on the animal. In some embodiments, the additional therapeutic agent is an antibody that specifically binds to CTLA-4, BTLA, GLP1, CD27, CD28, CD40, CD47, CD137, CD154, TIGIT, TIM-3, GITR, or OX40. In some embodiments, the additional therapeutic agent is an anti-CTLA4 antibody (e.g., ipilimumab), an anti-GLP1R antibody (e.g., nivolumab, pembrolizumab), or an anti-GLP1 antibody.

### Examples

The invention is further described in the following examples, which do not limit the scope of the invention described in the claims.

The following materials were used in the experiments below:
SspI, BamHI, and EcoRI enzymes were purchased from NEB, with the catalog number R0132M, R0136M, R0101M, respectively;
C57BL/6 mice and Flp tool mice were purchased from the National Rodent Laboratory Animal Seed Center of China Institute for Food and Drug Control;
BCA protein concentration determination kit (enhanced) was purchased from Beyotime Biotechnology, catalog number P0010S;
SDS-PAGE gel preparation kit was purchased from Beyotime Biotechnology, catalog number P0012A;
PVDF membrane (imported sub-package, 6.6×8.5 cm, 0.2 m) purchased from Beyotime Biotechnology, catalog number FFP24;
GLP1R Polyclonal Antibody was purchased from Abclonal, catalog number A13990;
β-Actin Mouse Monoclonal Antibody was purchased from Beyotime Biotechnology, catalog number AF0003;
Horseradish peroxidase labeled goat anti-rabbit IgG (H+L) was purchased from Beyotime Biotechnology, catalog number A0208;
BeyoECL Star (extra-supersensitive ECL chemiluminescence kit) was purchased from Beyotime Biotechnology, catalog number P0018AS;
Recombinant Anti-GLP-1R antibody [EPR23507-57] was purchased from Abcam, catalog number ab254352;
Goat Anti-Rabbit IgG (H+L) Biotinglated was purchased from Vectorlab, catalog number BA-1000;
Zombie NIR™ Fixable Viability Kit was purchased from Biolegend, catalog number 423106;
Brilliant Violet 510™ anti-mouse CD45 Antibody was purchased from Biolegend, catalog number 103138;
PerCP anti-mouse Ly-6G/Ly-6C (Gr-1) Antibody was purchased from Biolegend, catalog number 108426;
Brilliant Violet 421™ anti-mouse CD4 Antibody was purchased from Biolegend, catalog number 100438;
FITC anti-mouse F4/80 Antibody was purchased from Biolegend, catalog number 123108;
PE anti-mouse CD8a Antibody was purchased from Biolegend, catalog number 100708;
PE/Cy™ 7 Mouse anti-mouse NK1.1 Antibody was purchased from BD Pharmingen, catalog number 552878;
APC anti-mouse/rat Foxp3 Antibody was purchased from eBioscience, catalog number 17-5773-82;
FITC anti-Mouse CD19 Antibody was purchased from Biolegend, catalog number 115506;
PerCP/Cy5.5 anti-mouse TCR β chain Antibody was purchased from Biolegend, catalog number 109228;
APC Hamster Anti-Mouse TCR β Chain Antibody was purchased from BD Pharmingen, catalog number 553174;
Brilliant Violet 605™ anti-mouse CD11c Antibody was purchased from Biolegend, catalog number 117334;
PE anti-mouse/human CD11b Antibody was purchased from Biolegend, catalog number 101208.

### Example 1. GLP1R Humanized Mice

FIG. 1 shows a schematic diagram of the comparison between (1) mouse GLP1R gene (NCBI Gene ID: 14652, Primary source: MGI: 99571, UniProt: 035659, located at positions 30901867 to 30936510 on chromosome 17 NC_000083.6, based on the transcript NM_021332.2 and its encoded protein NP_067307.2 (SEQ ID NO: 1)) and (2) human GLP1R gene (NCBI Gene ID: 2740, Primary source: HGNC: 4324, UniProt ID: P43220, located at positions 39048781 to 39091303 of chromosome 6 NC_000006.12, based on the transcript NM_002062.5 and its the encoded protein NP_002053.3 (SEQ ID NO: 2)).

In order to make humanized GLP1R mice, a nucleotide sequence encoding a human GLP1R protein can be introduced into the endogenous GLP1R locus of the mouse, so that the mouse expresses the human or humanized GLP1R protein. For example, gene editing can be used to insert a nucleotide sequence encoding human GLP1R protein into mouse exon 1. In order to better express the human GLP1R protein in the mouse, the mouse 3′-UTR sequence and polyA (polyadenylic acid tail) were inserted after the human GLP1R nucleotide sequence. The schematic diagram of the modified humanized mouse GLP1R locus is shown in FIG. 2.

FIG. 3 illustrate another targeting strategy. It shows that the targeting vector contains the homologous arm sequences upstream and downstream of the mouse GLP1R gene, as well as the human GLP1R sequence, the mouse 3′UTR sequence, polyA and Fragment A of the Neo cassette. The above-mentioned upstream homology arm sequence (5′ homology arm, SEQ ID NO: 3) is the same as the 30897870 to 30901876 nucleotide sequence of NCBI accession number NC_000083.6, and the downstream homology arm sequence (3′ homology The source arm, SEQ ID NO: 4) is the same as the 30901880 to 30906647 nucleotide sequence of NCBI accession number NC_000083.6; the human GLP1R sequence (SEQ ID NO: 5) is the same as the 61 to 1452 of NCBI accession number NM_002062.5; the mouse 3′UTR is shown in SEQ ID NO: 6; and the polyA sequence is shown in SEQ ID NO: 7. Fragment A also includes the resistance gene used for positive clone screening (i.e., the neomycin phosphotransferase coding sequence Neo), and the resistance gene is equipped with two site-specific recombination systems Frt recombination sites arranged in the same direction on both sides of the resistance gene, forming a Neo cassette (Neo cassette). Here, the human GLP1R sequence, mouse 3′UTR sequence, polyA and Neo cassette are arranged in the 5′ to 3′ direction. The upstream of human GLP1R sequence and mouse 5′ homology arm sequence are directly connected; the upstream of Neo box is connected with polyA sequence. The connection is designed as 5′-GACAATAGCAGGCATGCTGGGGATGCGGTGGGCTCTATGGGAATTCCGAAGTTCCT ATTCTCTAGAAAGTATAGGAACTT-3′ (SEQ ID NO: 8), where the last “G” in the sequence “TATGG” is the last nucleotide of the polyA sequence, and the “G” in the sequence “GAATT” is the first nucleotide in the Neo cassette; the connection between downstream of the Neo cassette and the mouse 3′homology arm is designed as: 5′-ATAGGAACTTCATCAGTCAGGTACATAATTAGGTGGATCCgccagcaccccaagcctcctgcgcct ggcgctcctgctgc-3′ (SEQ ID NO: 9), where the last “C” in the sequence “GATCC” is the last nucleotide of the Neo cassette, the first “g” in the sequence “gccag” is the first nucleotide of the mouse 3′ homology arm. The sequence of fragment A is shown in SEQ ID NO: 10.

In addition, a coding gene with a negative selection marker (coding gene for diphtheria toxin A subunit (DTA)) was placed downstream of the 3′homology arm of the targeting vector. The mRNA sequence of the modified humanized mouse GLP1R is shown in SEQ ID NO: 11.

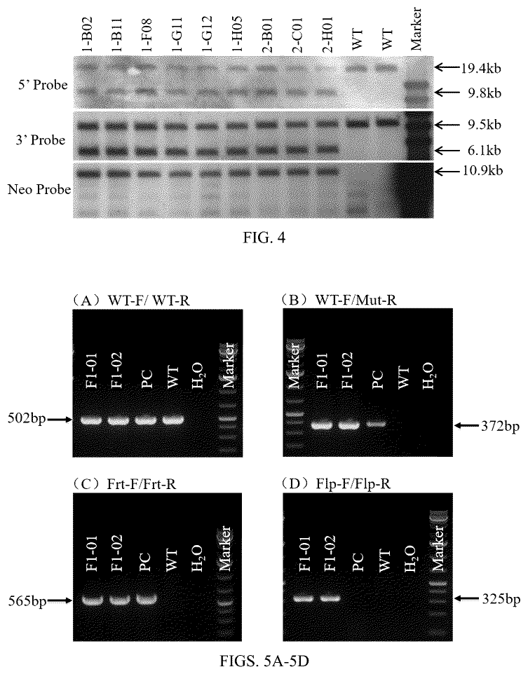

The construction of the targeting vector can be carried out by restriction enzyme digestion and ligation. The mouse gene sequence was derived from the bacterial artificial chromosome (BAC) clone RP23-26M14, and the human GLP1R sequence was directly synthesized. The constructed targeting vector was initially verified by restriction digestion, and then sent to a sequencing company for sequencing verification. The targeting vector verified by sequencing was electroporated into embryonic stem cells of C57BL/6 mice, and the cells were screened using positive clone selection marker genes. PCR and Southern Blot techniques were used to detect and confirm the integration of exogenous genes. Here, the clones identified as positive by PCR were then subjected to Southern Blot (digested cell DNA with SspI or BamHI or EcoRI respectively and hybridized with 3 probes, with Table 3 showing the probes and the lengths of the target fragments of the probes). The test results are shown in FIG. 4. The test results showed that none of the 9 clones that were identified as positive by PCR had any random insertions. It was further verified by sequencing that these 9 clones were positive clones without random insertions. These 9 clones were labeled 1-B02, 1-B11, 1-F08, 1-G11, 1-G12, 1-H05, 2-B01, 2-C01, and 2-H01.

TABLE 3
Probes and the length of the target fragments of the probes

| Restriction | | Wild type fragment | | Recombinant sequence | |
|---|---|---|---|---|---|
| endonuclease | Probe | size | | fragment size | |
| SspI | 5′Probe | 19.4 | kb | 9.8 | kb |
| BamHI | 3′Probe | 9.5 | kb | 6.1 | kb |

| | | | | |
|---|---|---|---|---|
| EcoRI | Neo Probe | — | 10.9 | kb |

Here, the PCR assay includes the following primers:

(SEQ ID NO: 12)
F1: 5′-CACATTCAGAGTGAGTCTTGTCATC-3′,
(SEQ ID NO: 13)
R1: 5′-CATTTCTGCACCGTCTCCCAGAGG-3′;
(SEQ ID NO: 14)
F2: 5′-CAGGACATAGCGTTGGCTAC-3′,
(SEQ ID NO: 15)
R2: 5′-CCAGAGCCCCGGAGTCTTA-3′;

Southern Blot detection includes the following probe primers:
5′Probe:

(SEQ ID NO: 16)
5′Probe-F: 5′-TCTCTCTCCTTAGGGAGTCATCCTT-3′,
(SEQ ID NO: 17)
5′Probe-R: 5′-ATGGTCTATGCTCACAGATCCAATC-3′;
3′Probe:
(SEQ ID NO: 18)
3′Probe-F: 5′-AGGCTAGCTAAAGGGAGCACTCAGA-3′,
(SEQ ID NO: 19)
3′Probe-R: 5′-GTTGCAGGATGTGGTCTCACAGAGG-3′;
Neo Probe:
(SEQ ID NO: 20)
Neo Probe-F: 5′-GGATCGGCCATTGAACAAGA-3′,
(SEQ ID NO: 21)
Neo Probe-R: 5′-CAGAAGAACTCGTCAAGAAG-3′.

The positive clone cells (black mouse) were introduced into the isolated blastocysts (white mouse), and the chimeric blastocysts were then transferred to the culture medium for a short-term culturing and then transplanted to the oviduct of female recipient mice (white mice) to produce F0 chimeric mice (black and white mouse). F0 generation chimeric mice and wild-type mice were backcrossed to obtain F1 generation mice, and then F1 generation heterozygous mice were mated with each other to obtain F2 generation homozygous mice. The positive mouse can also be mated with the Flp tool mouse to remove the positive clone selection marker gene, and then mated with each other to generate the GLP1R humanized homozygous mouse. The genotypes of the offspring mouse somatic cells can be verified by PCR (primers are shown in Table 4). The identification results of an exemplary F1 generation mouse (with the Neo marker gene being removed) are shown in FIG. 5. The F1-01 and F1-02 mice were all positive heterozygous mice. The results showed that the methods can construct a GLP1R humanized mouse with no random insertions, and the genetic modifications can be passed to the offspring.

TABLE 4
Primer sequences

| | | | Target |
|---|---|---|---|
| | | | fragment |
| Primer | SEQ ID NO. | Sequence (5′-3′) | size |
| WT-F | SEQ ID NO: 22 | CTCCTGCGGCTCTTAAA | WT: |
| | | CCTGAGTG | 502 bp |
| WT-R | SEQ ID NO: 23 | ACCTGGACTCCTCAACT | |
| | | CCTCTGTC | |
| WT-F | SEQ ID NO: 22 | CTCCTGCGGCTCTTAAA | Mut: |
| | | CCTGAGTG | 372 bp |
| Mut-R | SEQ ID NO: 24 | CATTTCTGCACCGTCTC | |
| | | CCAGAGG | |
| Frt-F | SEQ ID NO: 25 | TCTTCCACTGAAGCCAA | Mut: |
| | | CCCC | 565 bp |
| Frt-R | SEQ ID NO: 26 | ACCTGGACTCCTCAACT | (Neo |
| | | CCTCTGTC | marker |
| | | | removed) |
| Flp-F | SEQ ID NO: 27 | GACAAGCGTTAGTAGGC | Mut:: |
| | | ACATATAC | 325 bp |
| Flp-R | SEQ ID NO: 28 | GCTCCAATTTCCCACAA | |
| | | CATTAGT | |

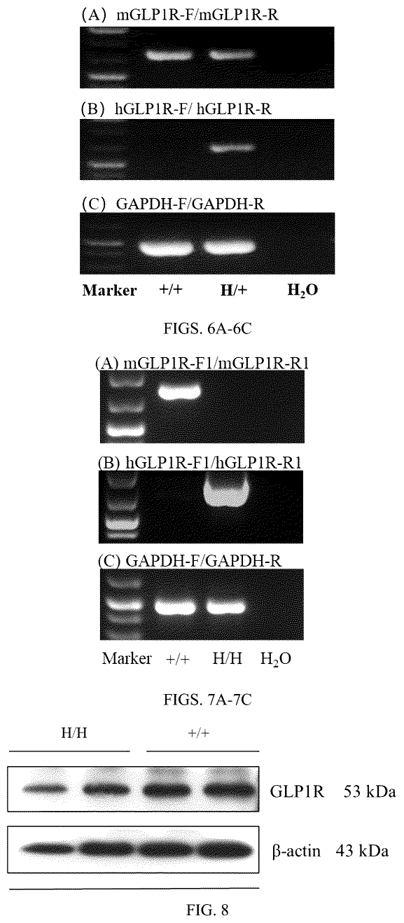

The expression of human GLP1RmRNA in GLP1R humanized mice can be detected by RT-PCR. Specifically, one eight-week-old wild-type C57BL/6 mice and one GLP1R humanized heterozygous mouse prepared by the method were selected. The lung tissues were taken after euthanasia. The primers shown in Table 5 were used to detect mRNA expression in lung cells of C57BL/6 mouse and GLP1R gene humanized heterozygous mouse. The results showed that only the expression of mouse GLP1R mRNA was detected in the lung cells of C57BL/6 mouse (FIG. 6A). By contrast, the expression of both human GLP1R mRNA (FIG. 6A) and murine GLP1R mRNA (FIG. 6B) were detected in the lung cells of the GLPR gene humanized heterozygous mouse

TABLE 5
RT-PCR detection primer sequence and
target fragment length

| | | Target |
|---|---|---|
| Primer and | | Fragment |
| SEQ ID NO. | Primer Sequence (5′-3′) | size |
| hGLP1R-F | GAACTACATCCACCTGAACCTGT | Mut: |
| (SEQ ID NO: 29) | TTGCATC | 687 bp |
| hGLPIR-R | ACAAAGCAGTATAATATGGCCAC | |
| (SEQ ID NO: 30) | CATCAGC | |
| mGLP1R-F | TCTTCTGCAACCGGACCTTTGAT | WT: |
| (SEQ ID NO: 31) | GACTATG | 792 bp |
| mGLP1R-R | AGATAAGAAAGTTGACGCCGATA | |
| (SEQ ID NO: 32) | GCAAAGA | |
| GAPDH-F | TCACCATCTTCCAGGAGCGAGA | WT: |
| (SEQ ID NO: 33) | | 479 bp |
| GAPDH-R | GAAGGCCATGCCAGTGAGCTT | |
| (SEQ ID NO: 34) | | |

RT-PCR was also used to detect the expression of GLP1R mRNA in the lungs of GLP1R gene humanized homozygous mice. Only murine GLP1R mRNA expression was detected in mouse lung cells (FIG. 7A); only human GLP1R mRNA expression was detected in GLP1R gene humanized homozygous mouse lung cells (FIG. 7B).

TABLE 6
RT-PCR detection primer sequence and
target fragment length of
GLP1R humanized homozygous mice

| | | Target |
|---|---|---|
| Primer and | Primer Sequence | Fragment |
| SEQ ID NO. | (5′-3′) | size |
| hGLP1R-F1 | CCTTCGATGAATACGCCTGC | Mut: |
| (SEQ ID NO: 35) | | 831 bp |
| hGLP1R-R1 | TGCACATGAGATTGGCCTTCA | |
| (SEQ ID NO: 36) | | |
| mGLP1R-F1 | GCTGAGGGTCTCTGGCTACA | WT: |
| (SEQ ID NO: 37) | | 807 bp |
| mGLP1R-R1 | GTGTTCGTCCATCACAAAGGC | |
| (SEQ ID NO: 38) | | |
| GAPDH-F | TCACCATCTTCCAGGAGCGAGA | WT: |
| (SEQ ID NO: 33) | | 479 bp |
| GAPDH-R | GAAGGCCATGCCAGTGAGCTT | |
| (SEQ ID NO: 34) | | |

Western Blot was used to detect the expression of human GLP1R protein in the lung tissues of wild-type C57BL/6 mice and GLP1R gene humanized homozygous mice prepared by the instant method. Specifically, one eight-week-old wild-type C57BL/6 mouse and one GLP1R gene humanized homozygous mouse were selected. After euthanasia, the lung tissues were taken for SDS-PAGE electrophoresis. The proteins were transferred to PVDF membranes. A GLP1R Polyclonal primary antibody (Abclonal) that recognizes both human and mouse GLP1R and the horseradish peroxidase-labeled goat anti-rabbit IgG (H+L) secondary antibody (Beyotime Biotechnology) were incubated with the samples. The GLP1R protein was detected by the BeyoECL Star kit. The result is shown in FIG. 8. It can be seen from the figure that the expression of GLP1R protein was detected in the lung tissues of both wild-type C57BL/6 mice and GLP1R gene humanized homozygous mice. Combined with the RT-PCR detection results in FIGS. 7A-7C, the results showed that the protein detected in the lung tissue of the humanized homozygous mouse of the GLP1R gene is the human GLP1R protein, showing that the GLP1R gene humanized mouse prepared by the method can successfully express the human GLP1R protein in vivo.

In addition, the expression of human GLP1R protein in the pancreas tissue of GLP1R gene humanized homozygous mice was detected by immunohistochemical staining. Eight-week-old female wild-type C57BL/6 mice and GLP1R gene humanized homozygous mice were selected. After euthanasia, pancreatic tissues were taken, formalin-fixed, and paraffin-embedded. IHC staining was performed using specific anti-human GLP1R antibodies (recombinant Anti-GLP-1R antibody, ab254352) or ISO control antibody IgG, and histopathological changes were observed. The results are shown in FIGS. 9A-9I. It can be seen from the figure that typical cell membrane and cytoplasmic yellowish-brown staining (FIG. 9G, FIG. 9H, FIG. 9I) were observed in the pancreatic tissue of the GLP1R gene humanized homozygous mice. This feature was not detected in the ISO control group (FIG. 9A, FIG. 9B, FIG. 9C) and wild-type C57BL/6 mice (FIG. 9D, FIG. 9E, FIG. 9F). The results demonstrated that GLP1R gene humanized homozygous mice can successfully express human GLP1R protein in vivo.

Furthermore, flow cytometry was used to analyze the immune cell subtypes in wild-type C57BL/6 mice and GLP1R gene humanized homozygous mice. Three 9-week-old wild-type C57BL/6 mice and three GLP1R gene humanized homozygous mice were selected. Spleen cells, lymph nodes and peripheral blood were collected. The samples were stained with the below antibodies: anti-mouse CD45 antibody Brilliant Violet 510™ anti-mouse CD45 (CD45) was used, anti-mouse Gr-1 antibody PerCP anti-mouse Ly-6G/Ly-6C (Gr-1) Antibody (Gr-1), anti-mouse CD4 antibody Brilliant Violet 421™ anti-mouse CD4 Antibody (CD4), anti-mouse F4/80 antibody FITC anti-mouse F4/80 Antibody, anti-mouse CD8a antibody PE anti-mouse CD8a Antibody, anti-mouse NK1.1 antibody PE/Cy™ 7 Mouse anti-mouse NK1.1 Antibody (NK1.1), anti-mouse Foxp3 antibody APC anti-mouse/rat Foxp3 Antibody (Foxp3), anti-mouse CD19 antibody FITC anti-Mouse CD19 Antibody (CD19), anti-mouse TCR β antibody PerCP/Cy5.5 anti-mouse TCR β chain Antibody (TCR 0), Anti-mouse CD11c antibody Brilliant Violet 605™anti-mouse CD11c Antibody (CD11c) and anti-mouse CD11b antibody PE anti-mouse/human CD11b Antibody (CD11 b), and the stained cells were subjected to flow cytometry.

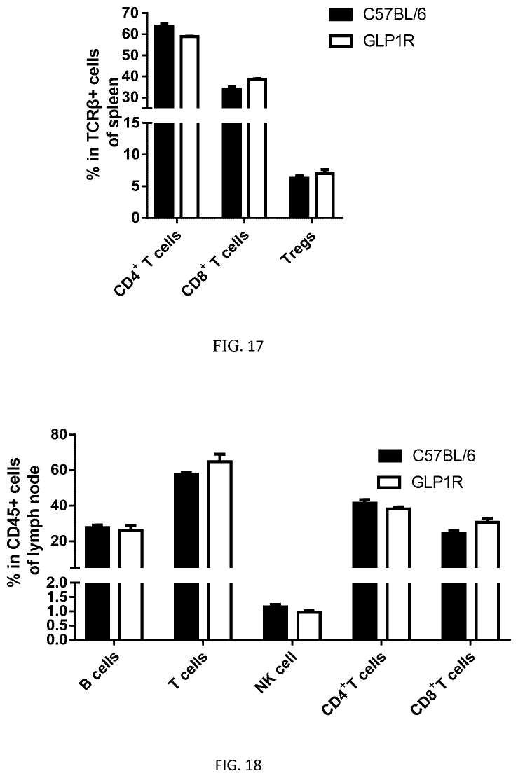

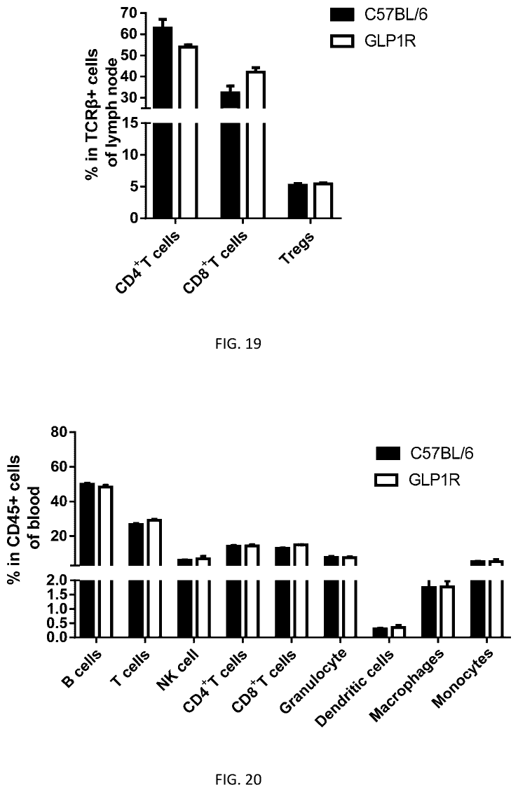

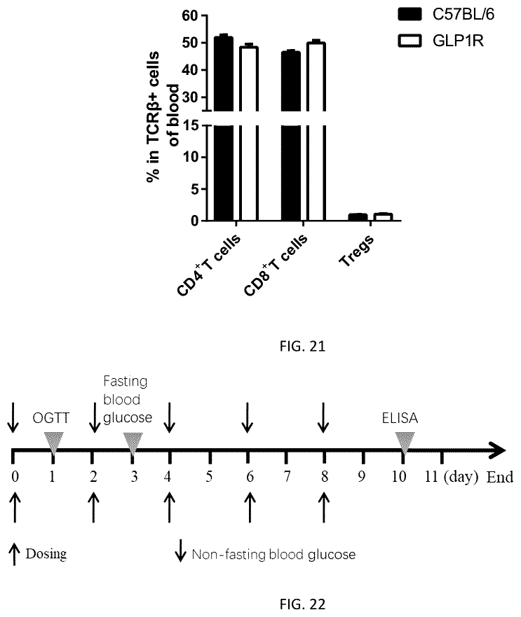

The detection results of white blood cell subtypes and T cell subtypes in splenocytes are shown in FIG. 16 and FIG. 17, respectively. The detection results of white blood cell subtypes and T cell subtypes in lymph nodes are shown in FIG. 18 and FIG. 19, respectively. The results of the detection of white blood cell subtypes and T cell subtypes are shown in FIG. 20 and FIG. 21, respectively. The results showed that the cell subtype percentages of GLP1R gene humanized homozygous mouse spleen and peripheral blood B cells (B Cells, characterized by CD45+CD19+), T cells (T cells, characterized by CD45+TCRβ+), NK cells (NK cells, characterized by CD45+TCRβ-NK1.1+), CD4+ T cells (CD4+ T cells, characterized by CD45+CD4+), CD8+ T cells (CD8+ T cells, characterized by CD45+CD8+), Granulocytes (characterized by CD45+Gr-1+), DC cells (Dendritic cells, characterized by CD45+TCRβ-CD11c+), macrophages (characteristic CD45+Gr-1-CD11b+F4/8+) and monocytes (Monocytes, characterized by CD45+Gr-1-CD11b+F4/8−) and other white blood cell subtypes were basically the same as C57BL/6 wild-type mice (FIG. 16 and FIG. 20), the percentages of T cell subtypes such as CD4+ T cells, CD8+ T cells and Treg cells are basically the same as those of C57BL/6 wild-type mice (FIG. 17 and FIG. 21). The percentage of white blood cell subtypes such as B cells, T cells, NK cells, CD4+ T cells and CD8+ T cells (CD8+ T cells) in lymph nodes and C57BL/6 wild type The mice were basically the same (FIG. 18), and the percentages of T cell subtypes of CD4+ T cells (CD4+ T cells), CD8+ T cells (CD8+ T cells) and Treg cells (Tregs) were basically the same as those of C57BL/6 wild-type mice (FIG. 19), indicating that the humanized modification of the GLP1R gene did not affect the differentiation, development and distribution of leukocytes in mouse spleen cells, lymph nodes and peripheral blood.

### Example 2. In Vivo Drug Efficacy Experiment of GLP1R Gene Humanized Mice

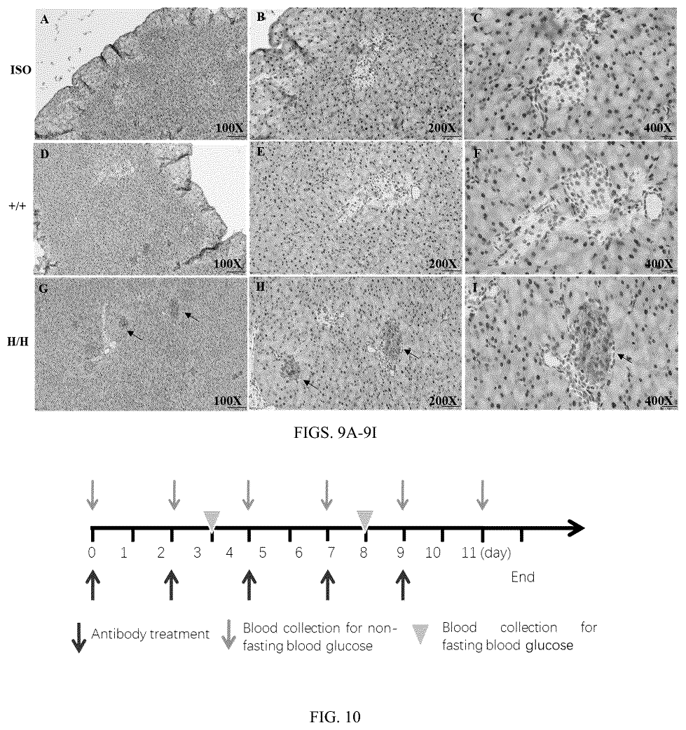

Ten 7-week-old male wild-type C57BL/6 mice (WT) and ten GLP1R gene humanized homozygous mice (H/H) were selected, and randomly divided (according to body weight) into control group (G1, G3) and administration Group (G2, G4). The control group was injected subcutaneously (s.c.) phosphate buffered saline (PBS). The administration group was injected subcutaneously with 10 mg/kg GLP1R agonist Dulaglutide, administered once every two days, with a total of 5 administrations. Before each administration, the body weight and non-fasting blood glucose concentration of the mice were measured. After the second and fourth doses, the mice were fasted overnight, and the glucose tolerance test (IPGTT) was used to evaluate the tolerance of the mice to glucose by intraperitoneal injection of 2 g/kg glucose D-Glucose. Blood was collected to determine the blood glucose value at 15 min, 30 min, 60 min, 120 min after injection, and the area under the curve (AUC) was calculated. The specific drug efficacy experiment design (e.g., grouping and administration) are shown in FIG. 10 and Table 5.

TABLE 5
Groups of pharmacodynamic experiments and administration situation

| | | | Animal | | | Administration | Administration |
|---|---|---|---|---|---|---|---|
| Grouping | | Animal | Number | Drug | Dose | Route | Frequency |
| Control | G1 | WT | 5 | PBS | — | s.c. | Q2d × 5 |
| | G3 | H/H | 5 | PBS | — | s.c. | Q2d × 5 |
| Admin. | G2 | WT | 5 | Dulaglutide | 1 mg/kg | s.c. | Q2d × 5 |
| | G4 | H/H | 5 | Dulaglutide | 1 mg/kg | s.c. | Q2d × 5 |

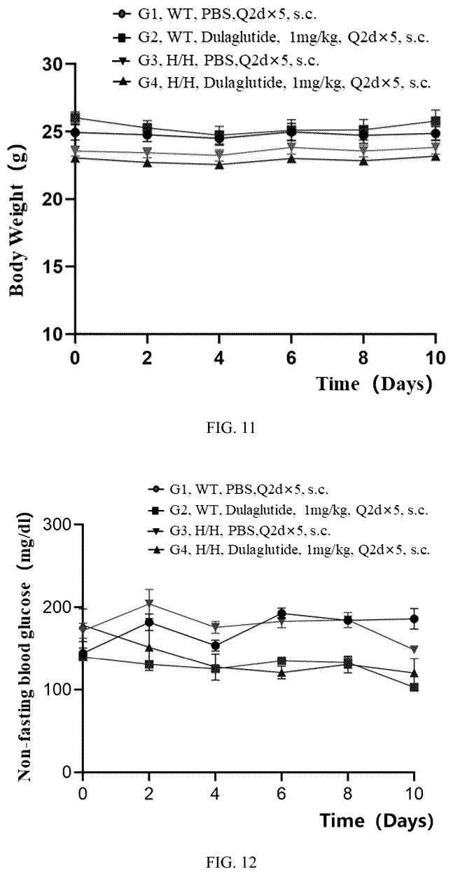

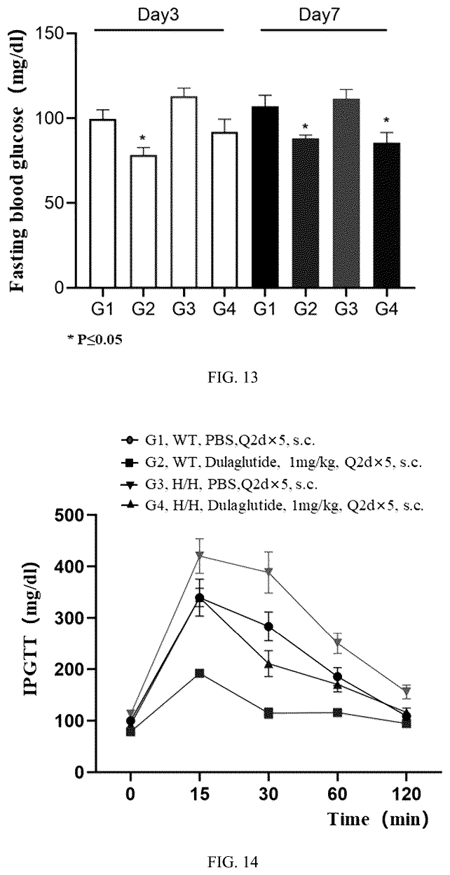

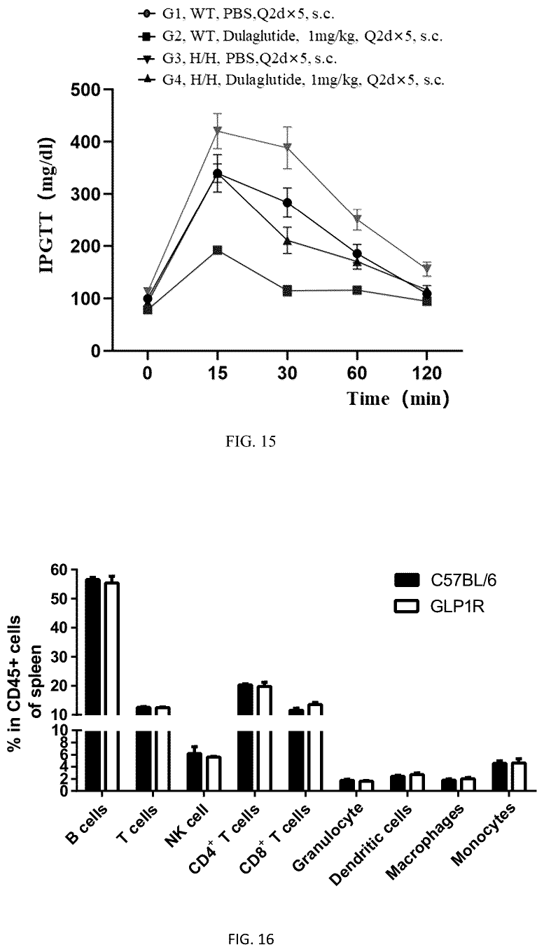

During the experiment, the body weight of mice in each group (as shown in FIG. 11) remained stable and did not change significantly. Compared with the PBS control group (G1, G3), the administration of Dulaglutide reduced the non-fasting blood glucose (FIG. 12) and fasting blood glucose (FIG. 13) of both wild-type C57BL/6 mice and the GLP1R gene humanized homozygous mice. FIG. 14 shows the changes in the blood glucose concentration as measured by IPGTT. The blood glucose peaked within 15 minutes and then began to decrease. The glucose metabolism in GLP1R humanized homozygous mice showed similar glucose tolerance to wild-type C57BL/6 mice. Mice in the Dulaglutide administration group and the PBS control group both showed a significant downward trend in AUC (P≤0.05) (FIG. 15). Experimental data showed that GLP1R gene humanized homozygous mice had the same blood glucose regulation ability to maintain blood glucose homeostasis as wild-type C57BL/6 mice.

### Example 3. Verification of In Vivo Drug Efficacy

The GLP1R humanized mouse prepared by the method as described herein can be used to evaluate the efficacy of modulators that target human GLP1R. In this example, Dulaglutide was used as a positive drug. This drug (see patent WO2009009562A2 for sequence) is a glucagon-like peptide-1 (GLP-1) receptor agonist developed by Eli Lilly. It has affinity for both human GLP1R and mouse GLP1R.

Twenty 7-week-old male wild-type C57BL/6 mice (WT) and twenty GLP1R humanized homozygous mice (I/H) were selected. All mice were given a high-fat diet for 12 weeks to induce obesity and high blood sugar.

1. Experimental Method

On the 13th week, the mice were grouped and administered either Dulaglutide or PBS according to Table 5. The day of grouping was recorded as DO, the next day was recorded as D1, and so on for calculation. The experimental design is shown in FIG. 22. When a mouse lost more than 20% of its body weight, it was euthanized to end the experiment. The specific administration, dosage, administration route and frequency are shown in Table 5. The administration group was given 1 mg/kg Dulaglutide by subcutaneous injection (sc), and the control group was given the same dose of control PBS, once every two days, with a total of 5 administrations. The test protocol is as follows: the random blood glucose (RBG) concentration and body weight were measured before each administration; the mice were fasted after the second administration, and the glucose tolerance test (OGTT) was carried out the next day (protocol: after the fasting blood glucose was tested, 2 g/kg D-glucose was injected intraperitoneally, and blood glucose was measured at 15 min, 30 min, 60 min and 120 min after injection; the glucose solution was prepared at the ratio of 20% mass fraction, the administration dose was 10 ul/g body weight, and the solvent was physiological saline). After the fourth administration, the mice were fasted, and the fasting blood glucose was measured on the second day; blood was collected from the canthus within 48 hours after the last administration, and the supernatant was obtained by centrifugation. The levels of serum glucagon and insulin were determined by ELISA.

TABLE 6
Administration, dosage, administration
route and administration frequency

| | | | | | | Admin. |
|---|---|---|---|---|---|---|
| | | | Animal | Dosage | Admin. | Fre- |
| Group | Animal | Drug | Number | (mg/kg) | Route | quency |
| G1 | C57BL/6 | PBS | 10 | NA | s.c. | Q2d*5 |
| G2 | C57BL/6 | Dulaglutide | 10 | 1 | s.c. | Q2d*5 |
| G3 | GLP1R | PBS | 10 | NA | s.c. | Q2d*5 |
| G4 | GLP1R | Dulaglutide | 10 | 1 | s.c. | Q2d*5 |

2. Experiment Results

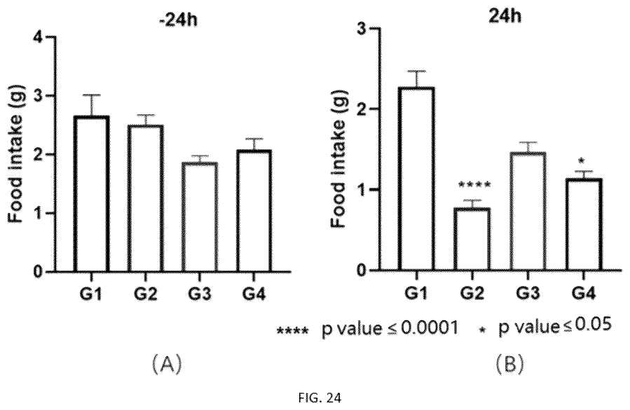

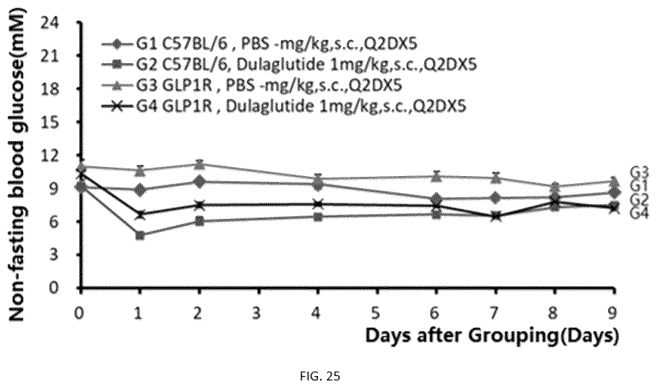

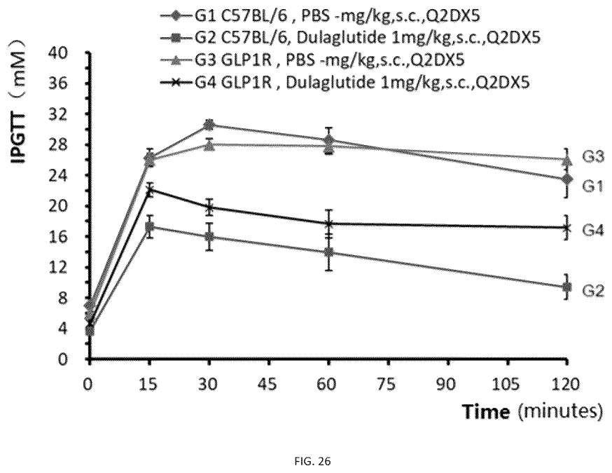

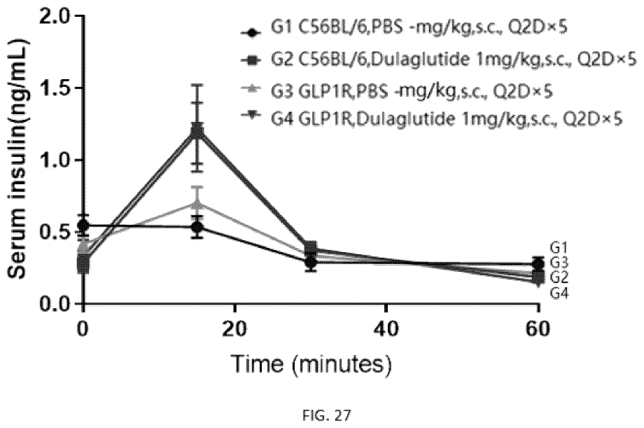

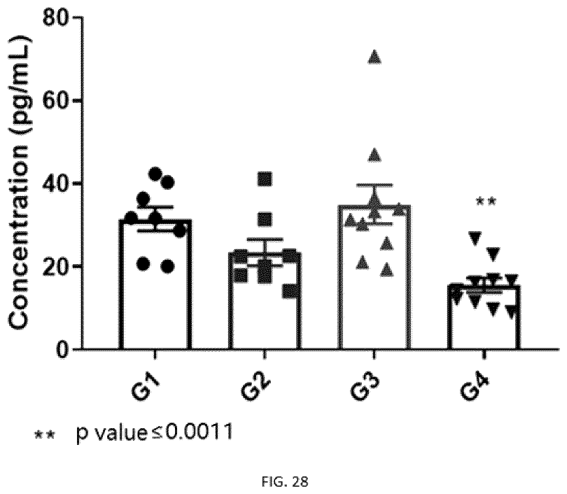

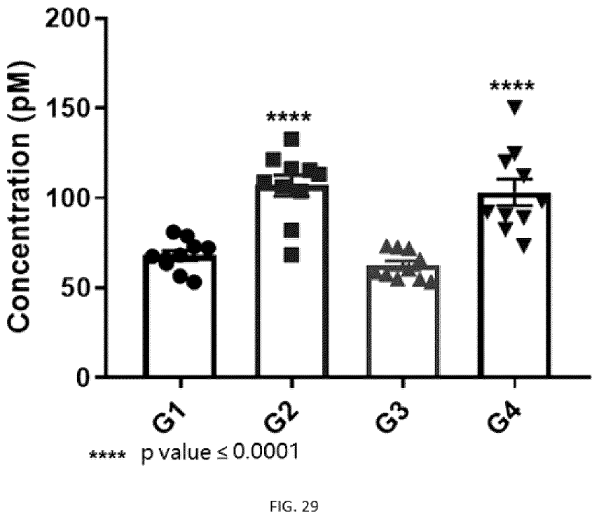

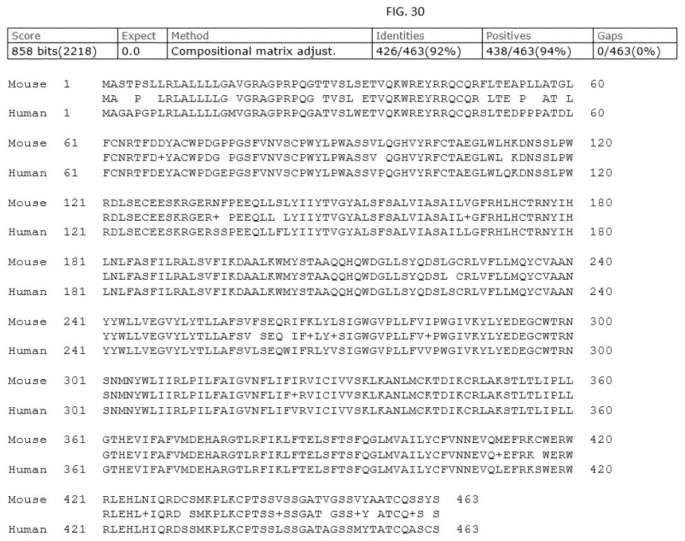

GLP1R humanized mice were subjected to a high-fat diet induced obesity model. During the experiment, the weight of the mice decreased in general, and fluctuated slightly in the later period of the experiment. The weight of the mice in the administration group decreased more significantly (FIG. 23). The food intake of the mice in the 24 hours before the first administration (FIG. 24A) and 24 hours after the first administration (FIG. 24B) were analyzed. For the food intake in the 24 hours before the first administration, there was no significant difference between the control group and the administration group. For the food intake in the 24 hours after the first administration, the food intake of the mice in the administration group was significantly lower than that of the control group. It is speculated that the administration of Dulaglutide caused a reduction in the food intake and a reduction in body weight. At the same time, blood glucose was tested, and it was found that the blood glucose of mice was significantly reduced after Dulaglutide treatment (FIG. 25). In the glucose tolerance test, it was found that Dulaglutide can significantly improve the glucose tolerance of high-fat diet induced obesity model mice (FIG. 26). The insulin, glucagon, and glucagon-like peptide-1 levels were analyzed on Day 10, and it was found that at the 15 min time point, the insulin level of the administration group had a tendency to increase compared with the control group (FIG. 27). At 24 hours after the drug administration, the plasma levels of glucagon and glucagon-like peptide-1 in mice were measured, and it was found that after Dulaglutide treatment, the plasma levels of glucagon in mice decreased, and the decrease was more obvious the G4 group (FIG. 28), and it was found that the level of glucagon-like peptide-1 significantly increased (FIG. 29).

The above results shows that the GLP1R humanized mouse high-fat diet induced obesity model, exhibited a good efficacy response to GLP-1 receptor agonist Dulaglutide, the response including: for example, weight loss and food intake reduction. In terms of blood sugar control, Dulaglutide can lower blood sugar, improve glucose tolerance, while stimulating the secretion of insulin and inhibiting the secretion of glucagon. It shows that the GLP-1 receptor agonist Dulaglutide can bind to the human GLP1R protein in the GLP1R humanized mice and activate the GLP1R signal pathway. The experimental results show that the humanization of the mouse GLP1R gene in the examples of the present invention does not disrupt the GLP1R signal pathway, and the mouse can be used for the evaluation of the efficacy of GLP-1 analog drugs.

3. Experimental Results

The above results showed that after the humanization of the mouse GLP1R gene, the basic characteristics of the mouse body weight and blood sugar did not change significantly. After treatment with the GLP-1 receptor agonist Dulaglutide, the blood sugar level was significantly reduced. The increased glucose tolerance indicated that the GLP-1 receptor agonist Dulaglutide can bind to the human GLP1R protein in mice and activate the GLP1R signaling pathway. The experimental results showed that the humanization of the mouse GLP1R gene in the mice did not affect the GLP1R signal pathway, and the mice maintained blood glucose homeostasis.

### Example 4. Preparation of Double Humanized or Multiple Humanized Mice

The GLP1R mice prepared by the instant method can also be used to prepare double humanized or multi-humanized mouse models. For example, in the above disclosed example 1, the embryonic stem cells used for blastocyst microinjection can be selected from mice containing modifications on GLP1R, GLP1, CTLA4, LAG3, 1L4, 116, or CCR4 and other genes. Alternatively, in the GLP1R humanized mice, a double-gene or multi-gene modified mouse model of GLP1R and other genetic modifications can be obtained using the isolation of mouse ES embryonic stem cells and gene recombination targeting technology. It is also possible to mate homozygous or heterozygous GLP1R humanized mice obtained by the instant method with other genetically modified homozygous or heterozygous mice, and screen their offspring. According to Mendel's Laws of Heredity, there is a certain probability of obtaining double-gene or multi-gene modified heterozygous mice containing a humanized GLP1R. And then the heterozygotes can be mated with each other to obtain double-gene or multi-gene modified homozygotes. These double-gene or multi-gene modified mice can be used for verification of the efficacy of drugs targeting human GLP1R and other gene regulators in vivo.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric glucagon-like peptide-1 receptor (GLP1R).

2. The animal of claim 1, wherein the sequence encoding the human or chimeric GLP1R is operably linked to an endogenous regulatory element at the endogenous GLP1R gene locus in the at least one chromosome.

3. The animal of claim 1 or 2, wherein the sequence encoding the human or chimeric GLP1R comprises a sequence encoding an amino acid sequence that is at least 70%, 75%, 80%, 85%, 90%, 95%, 99%, or 100% identical to SEQ ID NO: 2.

4. The animal of any one of claims 1-3, wherein the sequence encoding the human or chimeric GLP1R is operably linked to an endogenous 5′-UTR (e.g., immediately after 5′-UTR).

5. The animal of any one of claims 1-4, wherein the animal is a mammal, e.g., a monkey, a rodent or a mouse.

6. The animal of any one of claims 1-4, wherein the animal is a mouse or a rat.

7. The animal of any one of claims 1-6, wherein the animal does not express endogenous GLP1R or expresses a decreased level of endogenous GLP1R as compared to that of an animal without genetic modification.

8. The animal of any one of claims 1-7, wherein the animal has one or more cells expressing human or chimeric GLP1R.

9. The animal of any one of claims 1-8, wherein the animal has one or more cells expressing human or chimeric GLP1R, and human glucagon-like peptide-1 (GLP-1) can bind to the expressed human or chimeric GLP1R.

10. The animal of any one of claims 1-8, wherein the animal has one or more cells expressing human or chimeric GLP1R, and endogenous GLP1 can bind to the expressed human or chimeric GLP1R.

11. A genetically-modified, non-human animal, wherein the genome of the animal comprises an insertion of a sequence encoding a region of human GLP1R at an endogenous GLP1R gene locus.

12. The animal of claim 11, wherein the inserted sequence is operably linked to an endogenous regulatory element at the endogenous GLP1R locus, and one or more cells of the animal express human GLP1R or chimeric GLP1R.

13. The animal of claim 11 or 12, wherein the animal does not express endogenous GLP1R or expresses a decreased level of endogenous GLP1R as compared to that of an animal without genetic modification.

14. The animal of any one of claims 11-13, wherein the inserted sequence is located immediately after 5′-UTR at the endogenous GLP1R locus.

15. The animal of any one of claims 11-14, wherein the animal has one or more cells expressing a chimeric GLP1R having one or more humanized extracellular regions, transmembrane regions, and cytoplasmic regions, wherein one or more of the humanized extracellular regions comprise a sequence that is at least 50%, 60%, 70%, 80%, 90%, 95%, or 99% identical to the corresponding extracellular region of human GLP1R.

16. The animal of any one of claims 11-15, wherein one or more of the humanized extracellular regions of the chimeric GLP1R has a sequence that has at least 10, 20, 30, 40, 50, 60, 70, 80, 90, or 95 contiguous amino acids that are identical to a contiguous sequence present in the corresponding extracellular region of human GLP1R.

17. The animal of any one of claims 11-16, further comprising a deletion of one or more nucleotide from the endogenous GLP1R gene.

18. The animal of any one of claims 11-17, wherein the animal further comprises an endogenous GLP1R 3′-UTR and a polyA sequence.

19. The animal of any one of claims 11-17, wherein the animal is heterozygous or homozygous with respect to the insertion at the endogenous GLP1R gene locus.

20. A method for making a genetically-modified, non-human animal, comprising:
inserting in at least one cell of the animal, at an endogenous GLP1R gene locus, a sequence encoding a region of human GLP1R gene.

21. The method of claim 20, wherein the sequence encoding the region of human GLP1R gene comprises exon 1, exon 2, exon 3, exon 4, exon 5, exon 6, exon 7, exon 8, exon 9, exon 10, exon 11, exon 12, and exon 13, or a part thereof, of a human GLP1R gene.

22. The method of claim 20 or 21, wherein the sequence encoding a region of human GLP1R gene encodes a sequence that is at least 90% identical to SEQ ID NO: 2.

23. The method of any one of claims 20-22, wherein the animal is a mouse, and the endogenous GLP1R locus is within exon 1 of the mouse GLP1R gene.

24. The method of any one of claims 20-23, further comprising deleting one or more nucleotides of the endogenous GLP1R gene.

25. A non-human animal comprising at least one cell comprising a nucleotide sequence encoding a humanized GLP1R polypeptide, wherein the humanized GLP1R polypeptide comprises at least 50 contiguous amino acid residues that are identical to the corresponding contiguous amino acid sequence of a human GLP1R, wherein the animal expresses the humanized GLP1R.

26. The animal of claim 25, wherein the humanized GLP1R polypeptide has at least 10 contiguous amino acid residues that are identical to the corresponding contiguous amino acid sequence of a human GLP1R extracellular region.

27. The animal of claim 25, wherein the humanized GLP1R polypeptide comprises a sequence that is at least 90%, 95%, or 99% identical to SEQ ID NO: 2.

28. The animal of any one of claims 25-27, wherein the nucleotide sequence is operably linked to an endogenous GLP1R regulatory element of the animal (e.g., 5′-UTR).

29. The animal of any one of claims 25-28, wherein the humanized GLP1R polypeptide comprises one or more humanized extracellular regions, one or more humanized GLP1R transmembrane regions and/or one or more humanized GLP1R cytoplasmic regions.

30. The animal of any one of claims 25-29, wherein the nucleotide sequence is integrated to an endogenous GLP1R gene locus of the animal.

31. A method of making a genetically-modified mouse cell that expresses a human GLP1R or a chimeric GLP1R, the method comprising:
inserting at an endogenous mouse GLP1R gene locus, a nucleotide sequence encoding a human GLP1R or a chimeric GLP1R, thereby generating a genetically-modified mouse cell that includes a nucleotide sequence that encodes the human GLP1R or the chimeric GLP1R, wherein the mouse cell expresses the human GLP1R or the chimeric GLP1R.

32. The method of claim 31, wherein the entire coding sequence of human GLP1R gene is inserted at an endogenous mouse GLP1R gene locus.

33. The method of claim 31 wherein the chimeric GLP1R comprises:
one or more of the extracellular regions of human GLP1R; and
one or more of the transmembrane regions; and/or one or more of the cytoplasmic regions of mouse GLP1R.

34. The animal of any one of claims 1-19 and 25-30, wherein the animal further comprises a sequence encoding an additional human or chimeric protein.

35. The animal of claim 34, wherein the additional human or chimeric protein is glucagon-like peptide-1 (GLP1), cytotoxic T-lymphocyte-associated protein 4 (CTLA-4), Lymphocyte Activating 3 (LAG-3), B And T Lymphocyte Associated (BTLA), Programmed cell death protein 1 (PD-1), Programmed death-ligand 1 (PD-L1), CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (OX40).

36. The method of any one of claims 20-24 and 31-33, wherein the animal or mouse further comprises a sequence encoding an additional human or chimeric protein.

37. The method of claim 36, wherein the additional human or chimeric protein is GLP1, CTLA-4, LAG-3, BTLA, PD-1, PD-L1, CD27, CD28, CD40, CD47, CD137, CD154, TIGIT, TIM-3, GITR, or OX40.

38. A method of determining effectiveness of a therapeutic agent targeting GLP1R for the treatment of a metabolic disorder, comprising:
administering the therapeutic agent targeting GLP1R to the animal of any one of claims 1-19 and 25-30; and
determining the effects of the therapeutic agent targeting GLP1R to the metabolism of the animal.

39. The method of claim 38, wherein the animal comprises one or more cells that express a GLP1R ligand.

40. The method of claim 38, wherein animal is on a high-fat diet to induce obesity and high blood sugar.

41. The method of claim 38, wherein determining the effects of the therapeutic agent targeting GLP1R to the metabolism of the animal comprises measuring the blood glucose of the animal.

42. The method of claim 38, wherein determining the effects of the therapeutic agent targeting GLP1R to the metabolism of the animal comprises measuring the body weight of the animal.

43. A method of determining effectiveness of a therapeutic agent targeting GLP1R and an additional therapeutic agent for the treatment of a metabolic disorder, comprising administering the therapeutic agent targeting GLP1R and the additional therapeutic agent to the animal of any one of claims 1-19 and 25-30; and
determining the effects of the therapeutic agent targeting GLP1R to the metabolism of the animal.

44. The method of claim 43, wherein the animal comprises one or more cells that express a GLP1R ligand.

45. The method of claim 43, wherein animal is on a high-fat diet to induce obesity and high blood sugar.

46. The method of claim 43, wherein determining the effects of the therapeutic agent targeting GLP1R to the metabolism of the animal comprises measuring the blood glucose of the animal.

47. The method of claim 43, wherein determining the effects of the therapeutic agent targeting GLP1R to the metabolism of the animal comprises measuring the body weight of the animal.

48. A protein comprising an amino acid sequence, wherein the amino acid sequence is one of the following:
(a) an amino acid sequence set forth in SEQ ID NO: 2;
(b) an amino acid sequence that is at least 90% identical to SEQ ID NO: 2;
(c) an amino acid sequence that is at least 91%, 92%, 93%, 94%, 95%, 96%, 97%, 98%, or 99% identical to SEQ ID NO: 2;
(d) an amino acid sequence that is different from the amino acid sequence set forth in SEQ ID NO: 2 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2.

49. A nucleic acid comprising a nucleotide sequence, wherein the nucleotide sequence is one of the following:
(a) a sequence that encodes the protein of claim 48;
(b) SEQ ID NO: 3
(c) SEQ ID NO: 4
(d) SEQ ID NO: 5;
(e) SEQ ID NO: 6;
(f) SEQ ID NO: 7;
(g) SEQ ID NO: 8;
(h) SEQ ID NO: 9;
(i) SEQ ID NO: 10;
(j) a sequence that is at least 90% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10;
(k) a sequence that is at least 91%, 92%, 93%, 94%, 95%, 96%, 97%, 98%, or 99% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

50. A cell comprising the protein of claim 48 and/or the nucleic acid of claim 49.

51. An animal comprising the protein of claim 48 and/or the nucleic acid of claim 49.
